(19) United States
(12) Patent Application Publication
(10) Pub. No.: US 2007/0020243 A1
(43) Pub. Date: Jan. 25, 2007

(54) Methods and compositions related to modulating the extracellular stem cell environment

(75) Inventors: Shiladitya Sengupta, Waltham, MA (US); Ram Sasisekharan, Bedford, MA (US); Nishla Keiser, Cambridge, MA (US); David Eavarone, North Quincy, MA (US); Tanyel Kiziltepe Bilgicer, Cambridge, MA (US); Aarthi Chandrasekaran, Cambridge, MA (US); David A. Berry, Brookline, MA (US); Kristine Holley, Boston, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Massachusetts Institute of Technology, Cambridge, MA (US)

(21) Appl. No.: 11/332,766

(22) Filed: Jan. 12, 2006

Related U.S. Application Data
(60) Provisional application No. 60/643,458, filed on Jan. 12, 2005.
(60) Provisional application No. 60/644,468, filed on Jan. 14, 2005.

(51) Int. Cl. A61K 35/12; C12N 5/08; C12N 5/06

(52) U.S. Cl. 424/93.7; 435/325; 435/366

(57) ABSTRACT

This invention relates, in part, to methods and compositions that modulate the stem cell environment. More specifically, the invention relates, in part, to methods and compositions for modulating stem cell differentiation. Such modulation, in some aspects of the invention, is accomplished by agents that modulate glycosaminoglycans in the stem cell microenvironment (i.e., at or on the cell surface and/or in the extracellular matrix). Therefore, methods and compositions are provide for modulating glycosaminoglycan moieties, e.g., heparan sulfate glycosaminoglycan (HSGAG) moieties, in the microenvironment of stem cells. Methods and compositions for promoting or inhibiting embryonic stem cell differentiation (e.g., differentiation into endothelial cells) are also provided. This invention also relates, therefore, in part, to cell populations (e.g., endothelial cell populations or impoverished endothelial cell populations) that can be produced with the methods and compositions provided. Furthermore, the invention relates, in part, to tissues, and uses thereof, formed by the methods and compositions provided. Moreover, the invention also relates, in part, to methods of treatment using the methods and compositions provided.

### RELATED APPLICATIONS

This application claims the benefit under 35 U.S.C. §119 of U.S. provisional application 60/643,458, filed Jan. 12, 2005, and U.S. provisional application 60/644,468, filed Jan. 14, 2005, each of which is incorporated herein by reference in its entirety.

### FIELD OF INVENTION

This invention relates, in part, to methods and compositions that modulate the stem cell environment. More specifically, the invention relates, in part, to methods and compositions for modulating stem cell differentiation. Such modulation, in some aspects of the invention, is accomplished by agents that modulate glycosaminoglycans present at or on the stem cell surface and/or in the extracellular matrix. Therefore, methods and compositions are provide for modulating glycosaminoglycans (e.g., heparan sulfate glycosaminoglycans (HSGAGs)) in the microenvironment of stem cells. This invention also relates, in part, to cell populations and tissues that can be produced with the methods and compositions provided. Furthermore, the invention relates, in part, to methods of treatment using the methods and compositions provided herein.

### BACKGROUND OF THE INVENTION

The formation of new blood vessels is involved in many physiological processes such as reproduction, development tissue regeneration and wound healing. Under normal physiological conditions, formation of new blood vessels is highly regulated, so that it is turned on and turned off when necessary. However, many pathophysiological conditions are also associated with new blood vessel formation. For example, in some cases such as diabetes, hypercholesterolemia or advanced age, blood vessel formation is impaired as a result of endothelial cell dysfunction resulting in ischemic tissue (Rivard et al., Circulation, 1999, 99:111-120; Rivard et al., Am. J. Pathol., 1999, 154:355-363; Van Belle et al., Circulation, 1997, 96:2667-2674). For such cases, endothelial cell transplantation, regeneration and tissue engineering have potential therapeutic implications in treating patients. In contrast, in other pathophysiological cases, new blood vessel formation takes place in an unregulated, persistent manner. For example, in arthritis, new blood vessels invade the joint and cause the destruction of the cartilage, and in cancer, tumor cells continuously stimulate the growth of new blood vessels for the tumor itself to grow. Under these circumstances, inhibition of new blood vessel formation carries therapeutic implications in treating patients. Thus, methods that stimulate as well as methods that inhibit blood vessel formation have applications in treating human diseases.

Endothelial cells are integral components of blood vessels, and endothelial cell generation is a key step in new blood vessel formation. Thus, it is possible to regulate new blood vessel formation through regulating endothelial cell generation. Generation of endothelial cells happen by two mechanisms in mammals: through proliferation of pre-existing endothelial cells and through differentiation of progenitor stem cells. Several studies have demonstrated endothelial cell formation from endothelial progenitor stem cells (EPC) in vitro (Ishikawa et al., Stem Cells Dev., 2004, 13(4): 344-9; Iwaguro et al., Circulation, 2002, 105(6): 732-8). In addition, EPCs were shown to form new blood vessels in different animal models (Orlic et al., Ann N YAcad Sci., 2003, 996:152-7; Asahara et al., Circ. Res., 1997, 85:221-228; Kawamoto et al., Circulation, 2001, 103:634-637). However, EPCs represent only 0.1-0.5% of circulating blood cells, and they do not efficiently expand in culture, rendering their use in transplantation and regenerative therapies difficult.

Embryonic stem (ES) cells have also been studied and hold promise for use in tissue transplantation, regeneration and tissue engineering; however, the key limitation of their use in stem cell therapy lies in their potential to differentiate into different cell types in addition to the desired cell type. Upon differentiation, they often yield a combination of various cell types rather than a homozygous cell population of one type. Moreover, when injected into mice, ES cells can yield undesirable tumorigenic cell clusters called teratocarcinomas. For an effective therapeutic outcome, ES cell differentiation should be regulated to stimulate differentiation into the desired cell type. Although methods of regulating embryonic stem cell differentiation with bioactive materials (e.g., growth factors, proteoglycans or pituitary adenylate cyclase-activating polypeptide (PACAP)), special cultures, matrixes, or scaffolds have been discussed (See, e.g., U.S. Pat. Nos. 6,294,346; 5,851,832; 6,638,501; 6,399,369; 5,605,829 and 5,912,177; U.S. Patent Publications 20040092448, 20030175956, 20030224345, 20040126405 and 20040009589; and European Patent EP1452594), the structural content of HSGAGs or how they impinge on ES cell differentiation into endothelial cells has not been addressed.

### SUMMARY OF THE INVENTION

The invention relates, in part, to methods and compositions to modulate stem cell, e.g., embryonic stem (ES) cell, differentiation into cell types, e.g., endothelial cells, by modulating GAG moiety or moieties, e.g., HSGAG moiety or moieties, in the stem cell microenvironment. Modulation can also be accomplished, in some embodiments, by modulating cellular processes that affect the GAG moiety or moieties in the microenvironment. The methods and compositions that affect the GAG moieties can be biochemical, pharmacological or genetic in nature.

Therefore, in one aspect of the invention a method of modulating stem cell differentiation by contacting the microenvironment of a stem cell with a glycosaminoglycan (GAG)-modulating agent or a cell that expresses the GAG-modulating agent in an amount effective to modulate stem cell differentiation to endothelial cells is provided. The GAG-modulating agent can be any agent that results in the presence, absence or alteration of a glycosaminoglycan or the level at which the glycosaminoglycan is expressed in the stem cell microenvironment. In one embodiment, the GAG-modulating agent is a GAG-degrading agent. In another embodiment the GAG-degrading agent is a heparan sulfate glycosaminoglycan (HSGAG)-degrading agent. The HSGAG-degrading agent can be, but is not limited to, a bacterial HSGAG-degrading enzyme. In one embodiment the bacterial HSGAG-degrading enzyme is heparinase I, heparinase II, heparinase III, Δ4,5 glycuronidase, 2-O sulfatase, 3-O sulfatase, 6-O sulfatase or N-sulfatase or some combination thereof. In another embodiment the HSGAG-degrading agent is a mammalian HSGAG-degrading enzyme. In one embodiment the mammalian HSGAG-degrading enzyme is a/an heparanase, endoglucuronidase, sulfatase, acetyl transferase or N-acetylglucosaminidase or some combination thereof.

In another embodiment the GAG-modulating agent is a glycosaminoglycan. In one embodiment the glycosaminoglycan is a HSGAG. In another embodiment, the HSGAG is heparin, synthetic heparin, heparan sulfate, a low molecular weight heparin or a modified version thereof. In yet another embodiment the HSGAG is or comprises a highly sulfated disaccharide. In one embodiment the highly sulfated disaccharide is I/G-H_{NS,3S,6S}; I/G_{2S}-H_{NS, 3S}; I/G_{2S}-H_{NS,6S}; I/G_{2S}-H_{NH/Ac, 3S,6S}; or I/G_{2S}-H_{NS,3S,6S}. In still another embodiment the HSGAG is or comprises an undersulfated disaccharide. In yet another embodiment the undersulfated disaccharide is I/G-H_{NH/Ac}; I/G-H_{NS}; I/G-H_{NH,Ac,3S}; I/G-H_{NH/Ac,6S}; I/G-H_{NS,3S}; I/G-H_{NS,6S}; I/G-H_{NH/Ac,3S,6S}; I/G_{2S}-H_{NH/Ac}; I/G_{2S}-H_{NS}; I/G_{2S}-H_{NH/AC,3S}; or I/G_{2S}-H_{NH/Ac,6S}.

In one embodiment the cell that expresses the GAG-modulating agent expresses a glycosaminoglycan or an enzyme that is involved in GAG synthesis or degradation. In one embodiments the cell is engineered to express or to have altered expression of at least one GAG-modulating agent. In another embodiment the cell is engineered to express or overexpress at least one GAG-modulating agent. In still another embodiment the cell is engineered such that its expression of at least one GAG-modulating agent is inhibited. In yet a further embodiment the cell is engineered so that at least one GAG-modulating agent is expressed or overexpressed while the expression of at least one other GAG-modulating agent is inhibited. The GAG-modulating agent in these embodiments can be an enzyme involved in GAG synthesis (i.e., biosynthesis) or GAG degradation, for example. In one embodiment the GAG-modulating agent is a HSGAG-degrading enzyme. In another embodiment it is the stem cell that is altered to express or have altered expression of at least one GAG-modulating agent, and it is the agent (e.g., binding molecule, vector, etc.) that results in this alteration that is the GAG-modulating agent.

GAG-modulating agents includes any enzyme that alters or somehow modifies a glycosaminoglycan and/or affects its synthesis. Therefore, GAG-modulating agents can be any GAG biosynthetic or biodegradative enzyme. In one embodiment, the biosynthetic or biodegadative enzyme is a mammalian enzyme. In another embodiment, the biosynthetic or biodegradative enzyme is a/an glycosyltransferase, sulfotransferase, heparanase, endoglucuronidase, sulfatase, acetyl transferase or a N-acetylglucosamimidase. In one embodiment the sulfotransferase is N-deacetylase-N-sulfotransferase, 2-O heparan sulfate sulfotransferase, 3-O heparan sulfate sulfotransferase or 6-O heparan sulfate sulfotransferase. In another embodiment the endoglucuronidase is α-iduronidase or β-glucuronidase. In yet another embodiment the sulfatase is heparan-N-sulfatase, N-acetylglucosamine-6-sulfatase or N-acetylglucosamine-3-sulfatases. In still another embodiment the acetyl transferase is acetyl-coA:N-acetyltransferase.

It has been found that GAG-modulating agents can be used to inhibit or promote stem cell differentiation to endothelial cells. Therefore, methods and compositions are provided herein whereby stem cell differentiation to endothelial cells is inhibited with the use of a GAG-modulating agent. In another embodiment the GAG-modulating agent that inhibits stem cell differentiation to an endothelial cell is an HSGAG-degrading enzyme or some combination of HSGAG-degrading enzymes. In one embodiment the enzyme is heparinase I, heparinase III or a combination thereof. In another embodiment the inhibitor is an inhibitor of the GAG biosynthesis pathway (e.g., a molecule, such as an enzyme, involved in the biosynthesis of GAGs). In another embodiment the inhibitor is an inhibitor of an enzyme that promotes the sulfation of a HSGAG. In still another embodiment the inhibitor is an inhibitor of a sulfotransferase enzyme. In another embodiment embodiment the inhibitor is sodium chlorate. In a further embodiment the inhibitor can be an antibody or agent that binds to an enzyme involved in the biosynthesis of GAGs. In another embodiment the inhibitor can be a nucleic acid that binds to a nucleic acid that encodes (e.g., DNA or mRNA) an enzyme involved in the biosynthesis of GAGs.

Methods and compositions are also provided whereby stem cell differentiation to endothelial cells is promoted with the use of a GAG-modulating agent. In one embodiment the endothelial cell is an endothelial mammalian cell. In another embodiment the GAG-modulating agent that promotes stem cell differentiation to an endothelial cell is a glycosaminoglycan. In one embodiment the glycosaminoglycan is a HSGAG (e.g., heparin). In another embodiment the GAG-modulating agent is heparan sulfate. In still another embodiment the agent is a highly sulfated HSGAG. In still another embodiment, the HSGAG is or comprises a highly sulfated disaccharide. In yet another embodiment the GAG-modulating agent is an enzyme that promotes the sulfation of a HSGAG. In another embodiment, the enzyme is a sulfotransferase.

The methods provided herein can be in vivo methods or in vitro methods for modulating stem cell differentiation to endothelial cells, and there are many ways to contact the stem cell microenvironment with one or more GAG-modulating agents and/or one or more cells that express the GAG-modulating agents. For example, in in vitro methods, stem cells can be contacted with a GAG-modulating agent or a cell that expresses the GAG-modulating agent by adding the GAG-modulating agent or a cell that expresses the GAG-modulating agent to a culture of stem cells. In either in vitro or in vivo methods, for examples, GAG-modulating agents or a cell that expresses the GAG-modulating agent can be contacted with the stem cell microenvironment via a two or three dimensional structure to which the GAG-modulating agent orvcell that expresses the GAG-modulating agent is covalently or noncovalently bound thereto. The two or three dimensional structure is any structure to which a GAG-modulating or a cell that expresses the GAG-modulating agent can be bound. In one embodiment the structure is a scaffold. In another embodiment the structure is a matrix. In still another embodiment the structure is a support. In another embodiment the GAG-modulating agent or a cell that expresses the GAG-modulating agent is contacted with the stem cell microenvironment through a method of administration that places the GAG-modulating agent in contact with the in vivo stem cell microenvironment. In one embodiment the administration is systemic, local, topical or site-specific administration. In another embodiment the administration can be through the implantation or transplantation of a two or three dimensional structure to which the GAG-modulating agent or a cell that expresses the GAG-modulating agent is bound. In still another embodiment the administration is through site-specific implantation or transplantation. In still another embodiment the administration is intravenous or subcutaneous administration. In another embodiment the GAG-modulating agent or cell that expresses the GAG-modulating agent is bound to a targeting agents that targets the site in need of blood vessel formation or blood vessel formation inhibition. Such targeting agents can be binding proteins, such as antibodies, and will vary depending on the desired target site.

In one embodiment the stem cell microenvironment is in or near a site in need of blood vessel formation. In another embodiment the stem cell microenvironment is in or near a site in need of blood vessel formation inhibition. In still another embodiment the stem cell microenvironment is in or near ischemic tissue. In yet another embodiment the stem cell microenvironment is in or near a joint. In still another embodiment the stem cell microenvironment is in or near a wound. “In or near” as used herein is a location that is within or in close proximity to a certain site.

The methods and compositions provided herein can be used to affect the differentiation of any stem cell. In one embodiment, the stem cell is an embryonic stem (ES) cell. In another embodiment, the stem cell is a totipotent, pluripotent, hematopoietic, mesenchymal, neural or progenitor stem cells. In still another embodiment the stem cell is a mammalian stem cell. In one embodiment the mammalian stem cell is a human stem cell.

In another aspect of the invention a method of modulating stem cell differentiation by contacting the microenvironment of a stem cell with an agent that alters the biosynthetic or degradation pathway of the stem cell in an amount effective to modulate stem cell differentiation to endothelial cells is provided. In one embodiment the agent inhibits or promotes the presence of a GAG. In another embodiment, the agent is an inhibitor or activator of the GAG biosynthetic or degradation pathway. In still another embodiment, the agent is an inhibitor of the GAG biosynthetic pathway. In one embodiment, the inhibitor is sodium chlorate. In still another embodiment the inhibitor is an agent that inhibits the expression or function of a GAG biosynthetic enzyme (e.g., a sulfotransferase). In yet another embodiment the inhibitor is an antibody or nucleic acid. In another embodiment the agent can be any agent to results in genetic or protein expression alterations of the biosynthetic or degradation pathway of the stem cell, or other cells with which the stem cell microenvironment can be contacted. Therefore, methods and compositions for effecting genetic or protein expression alteration of one or more GAG-related genes are also provided. Such GAG-related genes include the genes responsible for the biosynthesis or degradation of GAGs as well as those that are associated with the GAG biosynthesis or degradation signaling pathway. Therefore, vectors, probes or other agents, e.g., antibodies, useful for modifying gene or protein expression are also considered to be GAG-modulating agents. The alterations provided ultimately lead to the expression, overexpression or inhibition (i.e., reduction or elimination) of at least one GAG-modulating agent.

In another aspect of the invention methods are provided for producing a population of cells by contacting the microenvironment of a stem cell with a GAG-modulating agent or a cell that expresses the GAG-modulating agent to inhibit or promote stem cell differentiation to endothelial cells, and obtaining a population of cells. In one embodiment the stem cell is promoted to differentiate to endothelial cells and the population of cells obtained is an endothelial cell population. In another embodiment, the endothelial cell population is a mammalian endothelial cell population. In still another embodiment the mammalian endothelial cell population is a human endothelial cell population. In one embodiment the stem cell is inhibited from differentiating to endothelial cells. In another embodiment the cell population obtained is impoverished of endothelial cells. In yet another embodiment the cell population obtained is enriched in muscle, neural or blood cells.

The contacting of the stem cell microenvironment with the GAG-modulating agent or a cell that expresses the GAG-modulating agent can be accomplished with either in vitro or in vivo methods. In one embodiment the contacting is accomplished by the addition of the GAG-modulating agent or a cell that expresses the GAG-modulating agent to a culture containing a stem cell. In another embodiment the stem cell microenvironment can be contacted with the a two or three dimensional support to which a GAG-modulating agent or a cell that expresses the GAG-modulating agent is bound.

Another aspect of the invention provides a composition comprising a cell population produced by a method provided herein. In one embodiment the cell population composition also comprises a pharmaceutically acceptable carrier.

In another aspect of the invention the cell population is used in tissue engineering. Therefore, compositions comprising a tissue containing a cell population produced by a method provided herein are provided. Methods of using a cell population produced by a method provided herein to engineer a tissue are also provided.

Any of the methods and compositions provided can be used for treatment purposes. In one aspect of the invention, a method of treatment that comprises administering a GAG-modulating agent, a cell that expresses the GAG-modulating agent, or a composition provided herein, to a subject in an amount effective to treat the subject is provided. In one embodiment the effective amount is an amount effective to promote or inhibit stem cell differentiation to endothelial cells. In another embodiment the subject is not otherwise in need of treatment with the GAG-modulating agent. In still another embodiment the subject is in need of blood vessel formation. In a further embodiment the subject is in need of blood vessel formation inhibition. In one embodiment the subject has cancer, and the GAG-modulating agent, cell that expresses the GAG-modulating agent or composition is in an amount effective to treat cancer. In still another embodiment the subject has a neurodegenerative disorder or nervous system injury, and the GAG-modulating agent, cell that expresses the GAG-modulating agent or composition is in an amount effective to treat the neurodegenerative disorder or nervous system injury. In another embodiment the subject has arthritis. In yet another embodiment the subject is in need of muscle cell, blood cell or neural cell generation. In still another embodiment the amount effective is an amount effective to inhibit stem cell differentiation to endothelial cells. In yet another embodiment the subject has a chronic wound, and the GAG-modulating agent, cell that expresses the GAG-modulating agent or composition is in an amount effective to treat the chronic wound. In another embodiment, the subject has ischemic tissue or an ischemic disease (e.g., ischemic tissue is present in the subject as a result of impaired blood vessel formation), and the GAG-modulating agent, cell that expresses the GAG-modulating agent or composition is in an amount effective to treat the ischemic disease. In one embodiment the subject has diabetes, coronary artery disease or hypercholesterolemia. In another embodiment the subject is of an advanced age. In another embodiment the amount effective is an amount effective to promote stem cell differentiation to endothelial cells. In still another embodiment the subject has a disease that can be treated by the generation of blood cells. Therefore, in one embodiment the methods provided herein can be used instead of or in conjunction with the administration of a blood transfusion to a subject. In one embodiment the subject is one who is in need of a blood transfusion. In another embodiment the GAG-modulating agent, cell that expresses the GAG-modulating agent or composition provided herein is administered to a joint in the subject. In still another embodiment the GAG-modulating agent, cell that expresses the GAG-modulating agent or composition is administered to an area with aberrant blood vessel formation. Such an area has abnormal blood vessel formation and may be in need of blood vessel formation or blood vessel formation inhibition.

In one aspect of the invention any of the methods provided herein further comprises assessing stem cell differentiation to endothelial cells. In one embodiment the assessing is accomplished by determining the expression of a stem cell marker (e.g., Oct-4). In another embodiment the assessing is accomplished by determining the expression of an endothelial cell marker (e.g., wVf, VEGF-R2, VE-cadherin, eNOS, Tie-2, etc.). In still another embodiment the expression of a MAPK factor, such as ERK (e.g., the phosphorylation of ERK can be assessed), is determined. In a further embodiment the expression of one or more markers is determined. Where the expression of more than one marker is determined, any combination of markers can be used. In one embodiment the expression of a marker is determined with an antibody. In another embodiment expression of a marker is determined with a nucleic acid probe. In still another embodiment the expression of a marker is determined with real-time PCR analysis.

In one embodiment the composition is a composition comprising a cell population produced by a method provided herein. In one embodiment the GAG-modulating agent, cell that expresses the GAG-modulating agent or composition inhibits stem cell differentiation to endothelial cells. In another embodiment the GAG-modulating agent, cell that expresses the GAG-modulating agent or composition promotes stem cell differentiation to endothelial cells. In another embodiment the GAG-modulating agent is a HSGAG-degrading enzyme. In still another embodiment the HSGAG-degrading enzyme is heparinase I, heparinase III or both. In another embodiment the GAG-modulating agent is sodium chlorate. In still a further embodiment the GAG-modulating agent is a HSGAG. In yet a further embodiment the GAG-modulating agent is a highly sulfated HSGAG. In yet another embodiment the GAG-modulating agent is heparin or heparan sulfate.

In one embodiment the subject is a mammal. In another embodiment the subject is a human. In another embodiment the subject is one otherwise not in need of the compositions and methods of treatment as provided herein. Such a subject is one that would not receive the compositions and treatments provided without the demonstration of the need for the modulation of stem cell differentiation to endothelial cells as provided herein.

The methods provided herein are not intended to be limited to the use of only one GAG-modulating agent or method of contacting the GAG-modulating agent with the stem cell environment. Methods whereby more than one GAG-modulating agent, more than one cell that expresses one or more GAG-modulating agents and/or more than one method of contacting the stem cell environment with a GAG-modulating agent or cell that expresses the GAG-modulating agent are provided.

The methods and compositions provided can further include one or more additional therapeutic agents.

In another aspect of the invention a method for the culture of stem cells is provided. In one embodiment the method includes the step of placing undifferentiated cells on or in a gelatin B coated culture container in the presence of FBS, beta-mercaptoethanol and pyruvate, and in the absence of LIF without further passaging for 7 to 15 days. In one embodiment the container is a culture dish. In another embodiment the method further comprises plating the cells at a concentration of 1.25×10⁵ cells/100 mm² dish. In still another embodiment the FBS is 15% FBS. In yet another embodiment the FBS is 15% Hyclone FBS. In still a further embodiment the beta-mercaptoethanol is 30 mM beta-mercaptoethanol. In yet another embodiment the sodium pyruvate is 1 mM sodium pyruvate. In still another embodiment one or more growth factors is added to the culture.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIG. 1 shows a schematic outlining the effects of HSGAGs on embryonic stem (ES) cell differentiation to endothelial cells.

FIG. 2 shows an outline of the effects of HSGAGs on embryonic stem cell differentiation to endothelial cells. Production of endothelial cells from endothelial progenitor cells has previously not been optimized given that these cells make up only 0.1-0.5% of circulating blood cells and exhibit slow expansion in vitro. Production of endothelial cells from embryonic stem cells results in a high proliferation rate of cells which can then be used in blood vessel engineering (e.g., to correct impaired neovascularization).

FIG. 3 shows a schematic outlining the steps of differentiation from ES cells to other cells.

FIG. 4 shows a schematic outlining the optimization of differentiation conditions of ES cells. Factors for transforming ES cells into endothelial cells in vitro are provided.

FIG. 5 provides data demonstrating that embryonic stem cells differentiate into endothelial cells. The embryoid bodies were cultured in leukemia inhibitoring factor (LIF)-free medium for defined time periods. The differentiation into endothelial cells was quantified using specific markers. FIG. 5A is a graph showing results of flow cytometry (FACs) analysis and shows the labeling for von Willebrand factor (vWF) in ES cells that differentiated into endothelial cells by day 7. FIG. 5B shows micrographs depicting confocal images of embryoid bodies which had been stained with an antibody against vWf (FITC labeled). The nuclei were counterstained with propidium iodide. Images were captured at a 512×512 resolution using a Zeiss LSM510 confocal microscope. Stereological analysis was performed for quantification. Significant differentiation into endothelial cells was evident by day 7, and the cells started forming tubes by day 10. FIG. 5C shows graphs representing real-time quantitative PCR results, which revealed the upregulation of different specific endothelial cell markers as the stem cells progressively differentiated. The y-axis represents relative mRNA expression levels. The x-axis represents time in days.

FIG. 6 shows data analyzing embryonic stem cell differentiation into endothelial cells. The differentiation of ES cells into endothelial cells was detected by using cell specific markers. vWF, VEGF-R2, VE-cadherin and eNOS were used as endothelial cell specific markers and Octamer-4 (Oct-4) was used as an ES cell specific marker. FIG. 6A shows graphs depicting a flow cytometry analysis of vWF at different stages of differentiation. FIG. 6B shows micrographs of confocal images of vWF and Oct-4 staining in differentiating ES cells. The y-axis represents time in days, and the x-axis represents type of staining. FIG. 6C shows graphs depicting real-time PCR data of VEGF-R2, VE-cadherin, eNOS and Oct-4 at different stages of differentiation. The relative mRNA levels are normalized to day 3, in which no significant differentiation was observed. Altogether, these results show that Oct-4 transcription and expression progressively diminishes with differentiation, while that of vWF, VEGF-R2, VE-cadherin and eNOS increases, suggesting efficient differentiation towards an endothelial cell population. The y-axis represents relative mRNA levels, and the x-axis represents time in days. Representative images are shown.

FIG. 7 shows a schematic providing methods that can be used to determine if HSGAG profiles change.

FIG. 8 shows a schematic providing methods that can be used to modulate HSGAGs and in turn to affect the differentiation of ES cells.

FIG. 9 provides capillary electrograms showing the changes in cell surface HSGAGs during differentiation. The data was obtained from a compositional analysis of cell surface HSGAGs isolated from cultured embryoid bodies at defined time points. The sugars were collected along with the protein cores through trypsin digestion and purified with an ion-exchange column. The purified GAGs were subjected to heparinase I and heparinase III digestion and analyzed using capillary electrophoresis. As shown in the images, there was a significant increase in the HSGAG signal as the cells differentiated, when normalized to the cell numbers. The x-axis represents time in minutes, and the y-axis represents absorbance (mAu).

FIG. 10 provides graphs representing data from a real-time PCR analysis demonstrating the increase in the expression of HSGAG synthesis enzymes as the embryonic stem cells progressively differentiated. The graphs show the relative transcriptional levels of 20-sulfotransferases (20ST), 30-sulfotransferases (30ST), 60-sulfotransferases (6-OST), N-deacetylase-sulfotransferases (NDST) and their isoforms at different stages of differentiation. The transcripts of these enzymes progressively increased as ES cells differentiated. The y-axes represent relative mRNA expression levels normalized to β-actin. The x-axes represent time in days. Data from representative experiments are shown.

FIG. 11 provides graphs representing data from a real-time PCR analysis measuring the expression levels of HSGAG enzymes on cells treated with heparinase I, heparinase III, NaClO₃, and NaClO₃ plus heparin. The y-axes represent relative mRNA expression levels normalized to β-actin. The x-axes represent time in days.

FIG. 12 provides results from a flow cytometry analysis of the effects of enzymatic or pharmacological modification of HSGAGs on the differentiation of ES cells into endothelial cells. FIG. 12A shows the effects of treatments through vWF staining at different stages of differentiation (at either day 3 or day 7). FIG. 12B provides a bar plot of the percentage of cells that stained positively for vWF in the flow cytometry experiment. The y-axis represents the percentage of vWF positive cells, and the x-axis represents the type of treatment used. Extracellular degradation of HSGAGs by heparinase I and heparinase III treatment, as well as inhibition of HSGAG biosynthesis by sodium chlorate, inhibits differentiation of ES cells into endothelial cells.

FIG. 13 shows the effect of heparinase and chlorate treatment on ES to endothelial cell differentiation. Treatments with heparinases cleave the HSGAGs at specific sites of sulfated residues, while chlorate treatment inhibits the synthesis of HSGAGs. FIG. 13A provides micrographs depicting confocal images of embryoid bodies which had been stained with an antibody against vWF (FITC labeled). The nuclei were counterstained with propidium iodide. Images were captured at a 512×512 resolution using a Zeiss LSM5510 confocal microscope. Treatment with heparinases or chlorate inhibits the differentiation, as summarized in the bar graph. The y-axis of the bar graph represents the precent vWF staining seen per view field, and the x-axis represents the various cell treatments. FIG. 13B is a graph showing the quantification of the same effect using FACS; the cells were labeled with vWF, an endothelial cell marker. The bar graph summarizes the quantification. The y-axis of the bar graph represents the vWF-positive staining seen per view field, and the x-axis represents the various cell treatments.

FIG. 14 shows the effect of glycome regulation on differentiation, and the involvement of signaling pathways. The results demonstrate the effect of HSGAG reconstitution on stem cell differentiation into endothelial cells. The embryoid bodies were cultured under a glycosaminoglycan (GAG) synthesis inhibited condition, as a result of the addition of chlorate. The addition of exogenous heparin reversed the chlorate-treated inhibition of differentiation into endothelial cells. FIG. 14A provides micrographs depicting confocal images of embryoid bodies, which had been stained with an antibody against vWF (FITC labeled). The nuclei were counterstained with propidium iodide. Images were captured at a 512×5 12 resolution using a Zeiss LSM510 confocal microscope. Treatment with heparinases or chlorate inhibited the differentiation, as summarized in the bar graph. The y-axis represents the percent vWF staining seen per view field, the x-axis represents the various cell treatments. FIG. 14B shows the results from Western blots investigating the possible involvement of the MAPK and Wnt pathways in the role of HSGAG ES to endothelial cell differentiation. Treatment with various enzymes or pharmacological modification consistently altered these two pathways, which was recovered by the exogenous addition of heparin. A bar graph summarizes the quantification of the ratio of pERK to ERK (based on densitometry of the Western blot data). The y-axis represents the pERK/ERK ratio, and the x-axis represents the different cell treatments.

FIG. 15 provides results from a confocal microscopy analysis of the effects of enzymatic or pharmacological modification of the HSGAGs on differentiation of ES cells into endothelial cells. FIG. 15A is a micrograph showing vWF staining. Extracellular degradation of HSGAGs by heparinase I and heparinase III treatment, as well as inhibition of HSGAG biosynthesis by sodium chlorate, inhibited differentiation of ES cells into endothelial cells as detected by vWF staining. FIG. 15B is a micrograph showing Oct-4 staining. Although differentiation towards endothelial cells was inhibited, overall differentiation still did proceed as evidenced by Oct-4 staining. FIG. 15C is a micrograph showing a reconstitution experiment using the addition of heparin. Addition of exogenous heparin to sodium chlorate treated ES cells reconstituted conditions that favor differentiation towards endothelial cells as detected by increased vWF staining.

FIG. 16 provides further confocal microscopy results.

FIG. 17 provides confocal microscopy micrographs showing vWF and Oct-4 expression in differentiating J1 mouse ES cells. Nuclei were counterstained with propidium iodide. The y-axes represent time in days, and the x-axes show the type of staining used.

FIG. 18 shows the elucidation of the signaling pathways modulated by HSGAGs. Specifically, the effects of HSGAG modulation on the MAPK pathway in differentiating ES cells are demonstrated. FIG. 18A provides results from Western blots performed with ERK and phospho-ERK antibodies. Western blots showed that treatment with heparinases or sodium chlorate inhibited the phosphorylation of ERK. This inhibition was reversed by the addition of exogenous heparin. FIG. 18B provides a bar plot that shows the ratio of pERK/ERK (y-axis) with different treatments α-axis). These results suggest that the MAPK pathway is involved in the differentiation of ES cells, and HSGAGs are modulators of this pathway.

FIG. 19 illustrates the effects of different glycome-modifying treatments (enzymatic or pharmacological modification of HSGAGs) on ES to endothelial cell differentiation. Provided is a series of graphs showing the results of a real-time PCR measurement of the increase in the expression of specific endothelial cell markers in embryoid bodies as the stem cells differentiated into endothelial cells under different treatment conditions. The y-axes represent relative mRNA expression levels. The x-axes represent time in days. Treatment with the enzymes or chlorate prevents differentiation, while the addition of exogenous heparin promotes the differentiation of stem cells to endothelial cells. For FIG. 19A the markers were Oct-4 and VEGF-R2. Oct-4 is a stem cell marker which goes down with differentiation. The treatments were unable to prevent differentiation. VEGF-R2 is an endothelial cell marker, which increases as differentiation proceeds in the vehicle-treated cells. However, treatment with the enzymes or the synthesis inhibitor, chlorate, prevented the increase. The effect of chlorate was reversed by the addition of exogenous heparin. For FIG. 19B the markers used were for eNOS and VE-cadherin, both of which are endothelial cell markers. Expression increases as differentiation proceeds in the vehicle-treated cells. However, treatment with the enzymes or the synthesis inhibitor, chlorate, prevented the increase. The effect of chlorate was reversed by the addition of exogenous heparin.

FIG. 20 provides a drawing of a cell depicting specific proteins and providing a some points about the HSGAG data.

FIG. 21 is a diagram providing some implications in regard to regenerative cell therapy and cancer therapy.

FIG. 22 provides results showing that embryonic stem cells differentiate into endothelial cells. Embryoid bodies formed by J1 embryonic stem cells were cultured in LIF-free medium for defined time periods. The differentiation into endothelial cells was quantified using specific markers. FIG. 22A provides micrograph depicting confocal images of embryoid bodies which had been stained with an antibody against vWF (FITC labeled). The nuclei were counterstained with propidium iodide. Images were captured at a 512×5 12 resolution using a Zeiss LSM510 confocal microscope. Stereological analysis was performed for quantification. The staining for Oct-4 progressively diminished with differentiation over time while significant differentiation into endothelial cells was evident by day 7. FIG. 22B provides results from a FACs analysis, which shows the labeling for vWF, an endothelial cell marker, in cells isolated from embryoid bodies that had been allowed to differentiate over 3 or 7 days. FIG. 22C provides results from a real-time quantitative PCR, which reveal the upregulation of different specific endothelial cell markers (VEGF-R2, VE-cadherin and eNOS), and the downregulation of the stem cell marker, Oct-4, as differentiation progresses. The data shown are mean SEM of 2 to 3 independent experiments. FIG. 22D shows a phase contrast image of embryoid bodies (inset). Images shown are representative random images. FIG. 22E provides capillary electrophoretograms showing the compositional analysis of cell surface HSGAGs isolated from the cultured embryoid bodies at defined time points. The sugars were collected along with the protein cores through trypsin digestion and purified with an ion exchange column. The purified GAGs were subjected to heparinase I and heparinase III digestion and analyzed with capillary electrophoresis. There was a significant increase in the HSGAG signal as the cells differentiated, when normalized to the cell numbers. The images and figures shown are representative of 2-3 independent experiments with replicates.

FIG. 23 illustrates the effect of enzymatic or pharmacological modification of the cell surface glycome on the differentiation of stem cells into endothelial cells. Treatments with heparinases cleaved the HSGAGs at specific sites of sulfated residues, while chlorate treatment inhibited the synthesis of HSGAGs. Provided are micrographs depicting confocal images of embryoid bodies which had been stained with an antibody against vWF (FITC labeled). The nuclei were counterstained with propidium iodide. Images were captured at a 512×512 resolution using a Zeiss LSM510 confocal microscope. Treatment with heparinases or chlorate inhibited the differentiation. The graph shows the stereological analysis of the confocal images, showing quantitatively that glycome modification inhibits the differentiation of embryonic stem cells into endothelial cells. The embryoid bodies were cultured under a GAG synthesis inhibited condition, as a result of the addition of chlorate. The addition of exogenous heparin reversed the chlorate treated inhibition of differentiation into endothelial cells.

FIG. 24 provides results from Western blots that show the involvement of the MAPK pathway in HSGAG ES to endothelial cell differentiation. Treatment with various enzymes or pharmacological inhibitor consistently altered the pathway, which was recovered by the exogenous addition of heparin. (U=undifferentiated, Hep1=heparinase I, Hep3=heparinase III, Chl=chlorate, Hep=heparin.)

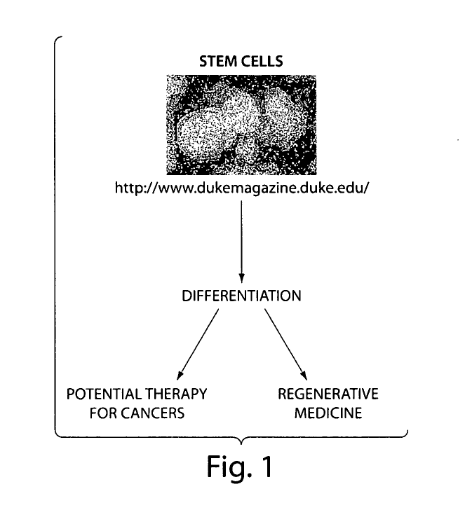

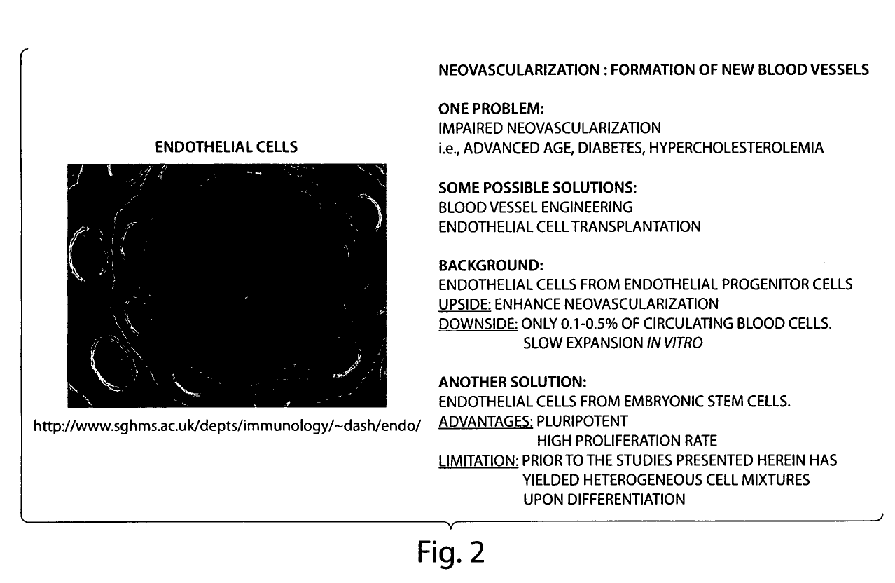

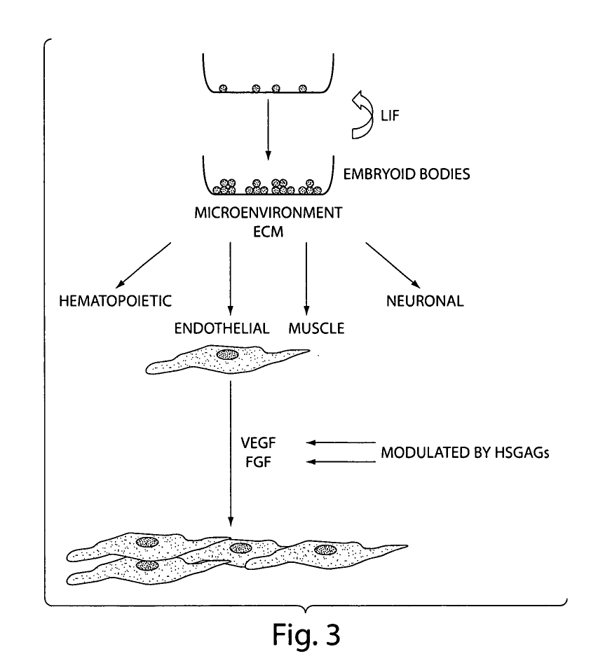

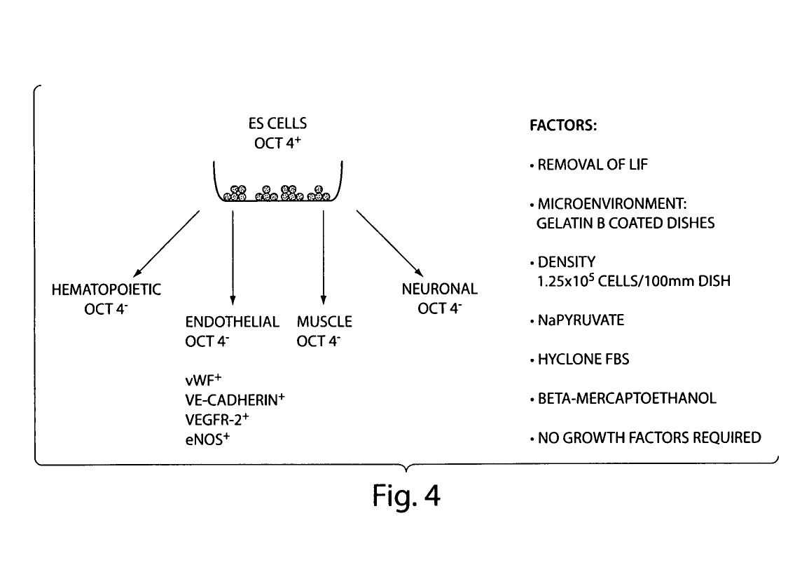

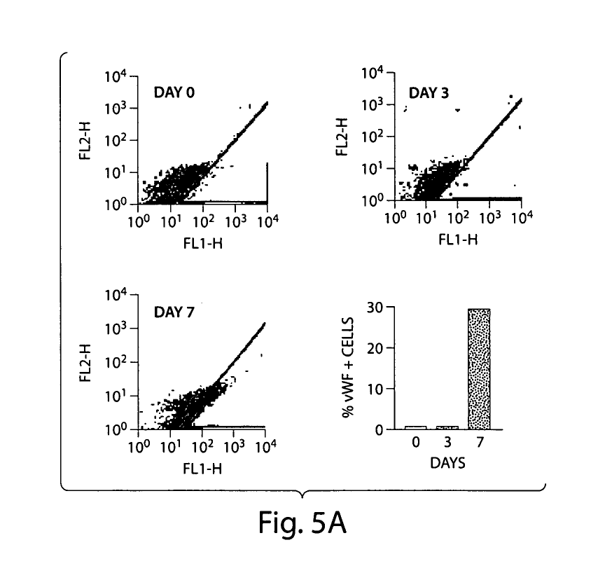

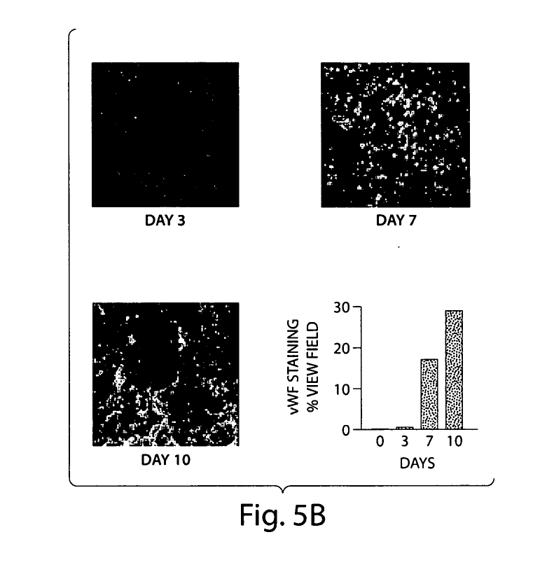

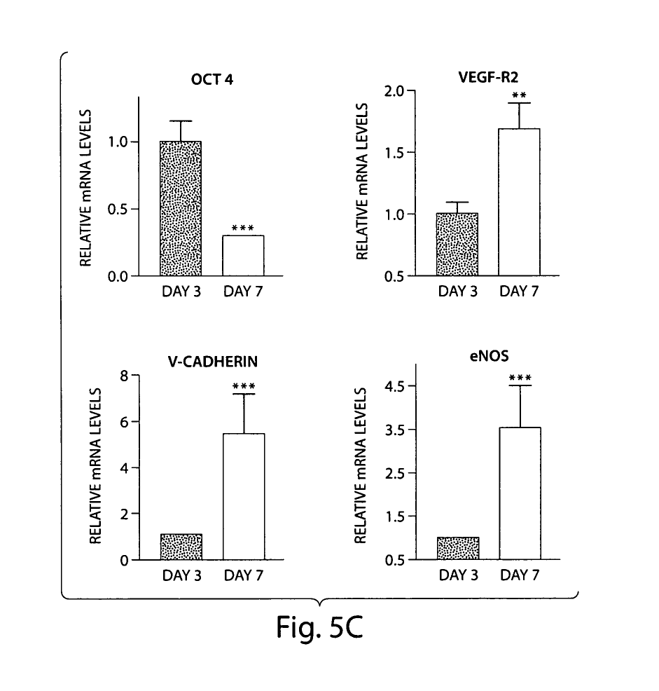

### DETAILED DESCRIPTION OF THE INVENTION

It has been found that stem cell differentiation can be regulated through the modulation of glycosaminoglycans. Therefore, methods and compositions to regulate stem cell differentiation through the modulation of glycosaminoglycans, e.g., heparan sulfate glycosaminoglycans (HSGAGs), are provided. Also provided are methods of treatment as well as methods and compositions directed to cell populations produced through the modulation of stem cell differentiation as provided herein.

One of the key components of the extracellular matrix (ECM) is a group of complex sugars called glycosaminoglycans (GAGs). GAGs, such as HSGAGs, are resident components of the ECM and form a major part of a cell's glycome. HSGAGs interact with numerous proteins and play a dynamic role in various cellular events such as proliferation, morphogenesis, adhesion, migration and cell death, tumor metastasis and neovascularization (Sasisekharan, R. and Venkataraman, G. (2000) Curr Opin Chem Biol 4, 626-31). Interestingly, although there are reports on the proteomal and transcriptomal analysis of stem cell differentiation (Brandenberger, R., et al. (2004) Nat Biotechnol 22, 707-16), no previous studies have been performed elucidating the role of the glycome in stem cell differentiation.

Using J1 mouse ES cells, it was demonstrated that as ES cells differentiate into endothelial cells, they lose their stem cell marker, Oct 4, and start expressing endothelial cell specific markers such as von Willebrand factor (vWf), VEGFR-2, Ve-cadherin and eNOS. Interestingly, as the ES cells differentiate, the HSGAG profile of cells changes drastically. HSGAGs were harvested at different differentiation stages of ES cells, and it was shown that the quantity of HSGAGs increased significantly as cells differentiate. Consistently, as measured by real-time PCR analysis, the HSGAG synthetic enzymes were also upregulated as ES cells differentiate. Therefore, provided herein are methods of modulating stem cell differentiation by contacting the microenvironment of a stem cell with a glycosaminoglycan (GAG)-modulating agent or a cell that expresses the GAG-modulating agent. As used herein, to “modulate stem cell differentiation” is intended to include inhibiting stem cell differentiation or promoting stem cell differentiation. To “inhibit stem cell differentiation” is to reduce the number of stem cells that undergo differentiation, slow the differentiation of stem cells, or stop one more stem cells from undergoing differentiation. To “promote stem cell differentiation” is to increase the number of stem cells undergoing differentiation or to speed up the differentiation process. In some embodiments, the modulation refers to the inhibition or promotion of the differentiation of one or more stem cells to one or more endothelial cells. The endothelial cells can be, for example, mammalian cells, and more specifically, they can be human endothelial cells.

The modulation of stem cell differentiation can be, but is not limited to, the modulation of the quality (i.e., structure; e.g., cleavage if the GAGs, changes to GAG sulfation or acetylation, etc.) and/or quantity of GAGs, such as HSGAGs, in the stem cell microenvironment. In a further embodiment methods and compositions for modulating the HSGAG moiety or moieties of the microenvironment to regulate stem cell differentiation are provided. The methods can be biochemical, pharmacological and genetic, and cause a change in the quantity or quality of HSGAGs of the microenvironment. These methods include exogenous and endogenous methods. Any combination of any endogenous and/or exogenous methods described herein can be used to modulate HSGAGs of the microenvironment to regulate stem cell differentiation.

As used herein, the “microenvironment of a stem cell” refers to the surface of one or more stem cells and/or the extracellular matrix of one or more stem cells. Therefore, the contacting of the microenvironment of the stem cell with a GAG-modulating agent or cell that expresses the GAG-modulating agent can be such that the GAG-modulating agent or cell that expresses the GAG-modulating agent is contacted with the stem cell surface, the extracellular matrix of the stem cell or both. Therefore, the stem cell microenvironment can be contacted with a GAG-modulating agent or cell that expresses the GAG-modulating agent in any way that introduces the GAG-modulating agent or cell that expresses the GAG-modulating agent to the microenvironment of at least one stem cell. This can be accomplished by, for example, adding one or more GAG-modulating agents or cells that express one or more GAG-modulating agents to a culture containing one or more stem cells, by introducing a two or three dimensional device to which at least one GAG-modulating agent or cell that expresses at least one GAG-modulating agent is bound to the stem cell microenvironment, or by some in vivo method of administration of one or more GAG-modulating agents or one or more or cells that express one or more GAG-modulating agents to a subject. Methods of administration to a subject include systemic, local, topical or site-specific administration. In some embodiments, the two or three dimensional device is administered by implantation or transplantation. Modification or alteration of the glycosaminoglycans in a specific subcompartment of the microenvironment (i.e. cell surface vs. extracellular matrix) may also be performed using the methods and compositions provided herein (e.g., to generate a shift in positive/negative GAG-mediated growth factor signaling).

The “GAG-modulating agent” is any agent that affects the presence, absence, kind or amount of at least one glycosaminoglycan. It has also been determined that differentiation of stem cells into endothelial cells can be inhibited by using inhibitors of GAG synthesis, such as sodium chlorate. Thus, stem cell differentiation can be regulated via modulating the GAG synthetic pathway. The term “GAG-modulating agent” includes agents that affect the synthesis or degradation of a glycosaminoglycan (e.g., enzymes involved in GAG biosynthesis or biodegradation, agents that affect the gene and/or protein expression of a molecule involved in the GAG biosynthesis or biodegradation pathway), agents that degrade glycosaminoglycans (e.g., GAG-degrading enzymes) and glycosaminoglycans themselves. Also included, therefore, are agents that inhibit the GAG biosynthetic pathway, such as sodium chlorate as well as heparan sulfate glycosaminoglycan-degrading enzymes.

As used herein a “GAG-degrading enzyme” or “HSGAG-degrading enzyme” is any enzyme that modifies, cleaves or somehow alters a glycosaminoglycan or heparan sulfate glycosaminoglycan, respectively. It is herein shown that differentiation of ES cells into endothelial cells is inhibited with HSGAG degrading enzymes, such as heparinase I and heparinase III, which degrade the HSGAGs of the microenvironment in a structurally specific manner. Intriguingly, there were differences in the levels of the inhibitory effect of heparinase I and heparinase III, and real-time PCR results indicated some orthogonal effects between heparinase I and III, suggesting that the structure (quality), as well as the quantity of HSGAGs impinge on the outcome of differentiation. Thus, stem cell differentiation can be regulated via qualitative and quantitative modulation of GAGs, by using biochemical methods such as GAG degrading enzymes or other degrading agents.

Glycosaminoglycans can be modified or altered, for example, by depolymerization, phosphorylation, sulfonation, regioselective sulfonation and/or desulfonation. GAG-degrading enzymes include but are not limited to, chondroitinases (e.g. chondroitinase AC, chondroitinase B, chondroitinase ABC), hyaluronate lyase, heparinases (e.g., heparinase I, heparinase II, heparinase II), keratanase, D-glucuronidase, L-iduronidase, glycuronidases (e.g., Δ4,5 glycuronidase), sulfatases (e.g., 2-O sulfatase, 3-O sulfatase, 6-O sulfatase), C5-epimerase, sulfotransferases, (e.g., 2-O sulfotransferase, 3-O sulfotransferase, 6-O sulfotransferase, and N-sulfotransferase (NDST)), modified versions, variants, functionally active fragments and combinations thereof. Examples of HSGAG-degrading enzymes include, for example, heparinase I, heparinase II, heparinase II1, Δ4,5 glycuronidase, 2-O sulfatase, 3-O sulfatase, 6-O sulfatase and N-sulfatase as well as modified versions, variants, functionally active fragments and combinations thereof.

Examples of enzymes that affect the biosynthesis or biodegradation of a glycosaminoglycan include, for example, glycosyltransferases, sulfotransferases, heparanases, endoglucuronidases, sulfatases, acetyl transferases and N-acetylglucosamimidases and modified versions and combinations thereof. Sulfotransferases include, for example, N-deacetylase-N-sulfotransferase, 2-O heparan sulfate sulfotransferase, 3-O heparan sulfate sulfotransferase and 6-O heparan sulfate sulfotransferase. Endoglucuronidases include, for example, α-iduronidase and β-glucuronidase. Sulfatases include, for example, heparan-N-sulfatase, N-acetylglucosamine-6-sulfatase and N-acetylglucosamine-3-sulfatases. Acetyl transferase include, for example, acetyl-coA:N-acetyltransferase.

The enzymes provided herein can be bacterial or mammalian enzymes. They can be produced from cell culture, such as from cultures of mammalian or bacterial cells, be recombinantly expressed or be synthesized with methods that are well known in the art. Flavobacteria synthesize many glycosaminoglycan-degrading enzymes as an integral part of their catabolic life cycle (Payza et al, J. Biol. Chem., 1956, 223, 853-858). An important class of enzymes that has been purified from flavobacteria and previously been used in elucidation of specific structure-function relationship of HSGAGs is the heparinases, including heparinases I, II and III (e.g. Venkataraman et al, Science, 1999, 286, 537-542; Dongfang Liu et al, Proc Natl Acad Sci USA, 2002, 99(2):568-573). The three heparinases are lyases, which cleave long chain HSGAGs to their dimeric structures leaving a Δ4,5 unsaturated uronidate in the non-reducing end. Each of the heparinases has its own unique HSGAG sequence at which it cleaves, making these enzymes valuable tools in obtaining sequence specific information. Heparinase I primarily cleaves HSGAGs at the highly sulfated regions such as -H_{NS,6X}-I_{2S}- linkage found primarily in heparin-like regions (Ernst et al., Crit, Rev. Biochem. Mol. Biol., 1995, 30, 387-444; Desai et al., Biochemistry, 1993, 32, 8140-8145; Jandik et al., Glycobiology, 1994, 4, 289-296). Heparinase III cleaves at undersulfated regions such as the H_{NAc}-I and H_{NY,6X}-G linkages which are the major disaccharides found in heparan sulfate (Ernst et al., Crit, Rev. Biochem. Mol. Biol., 1995; Linhardt et al., Biochemistry, 1990, 29, 2611-2617). Heparinase II is capable of recognizing and cleaving both sets of substrate linkages (Ernst et al., Crit, Rev. Biochem. Mol. Biol., 1995). Some other enzymes that flavobacteria synthesize to degrade HSGAGs in a sequence specific manner, which accordingly have potential uses in elucidation of specific structure-function relationship, are Δ4,5 glycuronidase, 2-O sulfatase, 3-O sulfatase, 6-O sulfatase and N-sulfatase.

Glycosaminoglycans can also be modified or altered by chemical agents. In particular, glycosaminoglycans can be modified with chemical degradation (e.g., periodate oxidation and base cleavage, alkaline degradation, nitrous acid cleavage). Therefore, chemical agents the can be used to modify or alter a GAG are also considered GAG-modulating agents.

The agents (e.g., chemical agents, enzymes, etc.), as provided herein, can be used to modify or alter glycosaminoglycans in a structurally specific manner. They can be used in any combination or in any order to effect the modification of one or more glycosaminoglycans. In one embodiment, these agents can be used to deplete the microenvironment of specific glycosaminoglycan structures (particular glycosaminoglycans, sequences or portions thereof). This can include the removal of degraded glycosaminoglycans away from the microenvironment and/or the destruction of a specific glycosaminoglycan structure. In another embodiment, these agents can be used to provide the microenvironment with specific glycosaminoglycan structures. This can include the production of a specific glycosaminoglycan structure and/or the maintenance of the structure in the stem cell microenvironment.

In addition, it was demonstrated that under conditions where the microenvironment lacks the structurally right composition of GAGs (e.g., generated by inhibiting HSGAG synthesis), supplementing the microenvironment with the structurally right composition of exogenous GAGs (e.g., heparin) induced stem cell (ES cell) differentiation to endothelial cells. Therefore, GAG-modulating agents can also be glycosaminoglycans. There are a number of glycosaminoglycans known in the art. Members of the glycosaminoglycan (GAG) family of complex polysaccharides includes dermatan sulfate (DS), chondroitin sulfate (CS), heparin/heparan sulfate (HSGAG), keratan sulfate and hyaluronic acid. The term “glycosaminoglycan” also refers to sulfated or highly sulfated glycosaminoglycans. Other examples of glycosaminoglycans include sulfated hyaluronic acid, heparan sulfate glycosaminoglycans (HSGAGs), biotechnologically prepared heparin, chemically modified heparin, synthetic heparin, heparinoids, enoxaparin, low molecular weight heparin (LMWH), or specific kinds of chondroitin sulfate, such as chondroitin sulfate A, chondroitin sulfate B or chondroitin sulfate C. Glycosaminoglycans also include modified versions of the glycosaminoglycan members provided herein as well as any other members of the glycosaminoglycan family known to those of ordinary skill in the art. Glycosaminoglycans, in some embodiments, include heparin-like polyanions which are similar to heparin and are naturally occurring or synthetic. Such heparin-like polyanions include poly(vinyl sulfate) and poly(anethole sulfonate). Glycosaminoglycans also include glycosaminoglycans that are di-, tetra-, hexa-, octa- or longer polysaccharide units. “Polysaccharide” is intended to refer to any polymer with two or more consecutively linked monosaccharide units.

Heparan sulfate glycosaminoglycans include many of the glycosaminoglycans already provided. HSGAGs include, for example, heparin, heparan, low molecular weight heparin, synthetic heparin, biotechnologically derived heparin, modified versions of the foregoing, etc. HSGAGs also includes those which are or comprise highly sulfated or undersulfated disaccharides. HSGAGs can be any polysaccharide that comprises any combination of the 32 possible disaccharide units.

HSGAGs are chemically complex and heterogeneous polysaccharides made up of a long chain of disaccharide repeat units consisting of an uronic acid [α-L-iduronic acid (I) or β-D-glucuronic acid (G)] linked 1,4 to an α-D-hexosamine (H) (Linhardt et al, 1991, Chem. Ind., 2:45-50; Casu et al, 1985, Adv. Carbohydr. Chem. Biochem., 43:51-134.) HSGAGs can vary in terms of the number of disaccharide repeat units as well as the chemical modifications internal to each repeat unit. The chemical modifications that can take place physiologically are sulfation at the 2-O carbon of the uronic acid and sulfation at the 3-O and 6-O carbons of the hexosamine, as well as N—H(NH₂), N-sulfation or N-acetylation of the hexosamine. Together, the four different modifications give rise to 2⁴=16 different possible structures for a disaccharide repeat with a particular uronic acid isomer. Since there are two uronic acid isomers: I and G, there could be 16×2=32 different plausible disaccharide units for HSGAGs. Combinations of the 32 building blocks yield tetra-, hexa-, or longer polysaccharide units with demonstrated biological significance (Venkataraman et al., Science, 1999, 286, 537-542). Out of the 32 possible disaccharide structures, the structures with chemical modification (sulfation) in 3 or 4 sites can be considered as the highly sulfated disaccharides, while the structures with 0, 1 or 2 sulfated sites are the less sulfated ones. Sequential combinations of these disaccharides can then result in regions of high and low sulfation within the HSGAG polysaccharide chain. Accordingly the following disaccharides can be categorized as highly sulfated: I/G-H_{NS,3S,6S}; I/G_{2S}-H_{NS, 3S}; I/G_{2S}-H_{NS,6S}; I/G_{2S}-H_{NH/AC,3S,6S}; and I/G_{2S}-H_{NS,3S,6S}, and the following disaccharides can be categorized as under-sulfated: I/G-H_{NH/Ac}; I/G-H_{NS}; I/G-H_{NH/Ac,3S}; I/G-H_{NH/Ac,6S}; I/G-H_{NS,3S}; I/G-H_{NS,6S}; I/G-H_{NH,Ac,3S,6S}; I/G_{2S}-H_{NH/Ac}; I/G_{2S}-H_{NS}; I/G_{2S}-H_{NH/Ac,3S}; and I/G_{2S}-H_{NH/Ac,6S}.

Unlike other structurally complex charged biopolymers such as DNA or RNA, HSGAGs do not undergo template-based biosynthesis. Instead, biosynthesis in mammals is regulated by a complex series of enzymatic interactions initiated within the Golgi apparatus. HSGAGs are attached to their core protein at a serine reside via tetrasaccharide linkage regions consisting of Glucuronic Acid-Galactose-Galactose-Xylose. After the initial formation of this linkage tetrasaccharide, the alternating addition of glucuronic acid and N-acetyl-glucosamine from their UDP-sugar nucleotide precursors forms a repeating 1,4-linked disaccharide chain. The disaccharide chain is further modified by a series of sulfotransferases, of which N-deacetylase-N-sulfotransferase (NDST) and the 2-O, 3-O, and 6-O heparan sulfate sulfotransferases play a key role. Tissue and substrate specific isoforms of each of these sulfotransferases have been discovered, indicating a further level of complexity in the biosynthesis of HSGAGs (Habuchi et al, Biochim Biophys Acta, 2000, 1474 (2), 115-127; Lindahl et al, J Biol Chem 273, 1998, 273 (39), 24979-24982; Sasisekharan, et al, Curr Opin Chem Biol, 2000, 4 (6), 626-631). Synthesized HSGAG structures can be modified through removal of sulfates via these sulfatase enzymes.

A cell's HSGAG composition is further regulated by a series of enzymes involved in the chemical degradation of the polysaccharide chain. First, the long carbohydrate chain is cleaved into smaller polysaccharide fragments by endoglycosidases termed heparanases. Heparanase expression has been implicated in a variety of physiological and pathological processes including cancer progression, angiogenesis, and development (Vlodavsky et al, Semin Cancer Biol, 2002, 12(2), 121-9). The remaining polysaccharide fragments are sequentially degraded by cleavage at the terminal end by an endoglucuronidase (either α-iduronidase or β-glucuronidase) following by desulfation of this residue via an epimer-specific sulfatase. The resultant terminal glucosamine is cleaved next by α-N-acetylglucoamimidase, following desulfation and N-acetylation of this residue by a combination of sulfatases and acetyl transferases of which heparan-N-sulfatase, acetyl-coA:N-acetyltransferase, N-acetylglucosamine-6-sulfatase, and N-acetylglucosamine-3-sulfatase are included. These enzymes are, therefore, also considered GAG-modulating agents.

Also included as GAG-modulating agents are agents that affect the gene and/or protein expression of molecules involved in the GAG biosynthesis or biodegradation pathway, such as the enzymes provided above. The reduction or elimination of the expression level or function of a gene and/or its protein can be accomplished using a variety of agents. It will be apparent to one of ordinary skill in the art that agents that reduce or eliminate the expression level or function of include binding molecules, such as antisense oligonucleotides (e.g., the antisense oligonucleotides of Bredesen et al., U.S. Pat. No. 5,324,654), RNAi molecules, binding polypeptides, e.g., antibodies or antibody fragments, intrabodies, small molecules or any other compound that binds and inhibits expression and/or function. Binding molecules may be isolated from natural sources or synthesized or produced by recombinant means. Methods for preparing or identifying molecules which bind to a particular target are well-known in the art. Binding polypeptides, such as antibodies, may easily be prepared by generating antibodies to a protein (e.g., the enzymes described herein) (or obtained from commercial sources) or by screening libraries to identify binding peptides or other binding compounds.

As provided herein, GAGs themselves are also considered GAG-modulating agents. GAGs can be produced with a number of well-known methods. A few of which are briefly described herein. GAGs can be produced through synthetic methods, by harvesting from the surface of GAG-expressing cells (e.g., purified from the original cells, such as mammalian cells, Flavoheparinum, etc.), and by recombinant methods (e.g., genetically engineered cells). Methods for producing the GAGs as provided herein can also include the use of GAG-degrading enzymes, chemical agents, proteases, etc. or any combination thereof. Chemical agents that can be used to harvest GAGs from cells include, for example, salts, acids, bases or detergents. For instance, GAG-degrading enzymes can be used to harvest GAGs of a specific structure from cells. As another examples, proteases can be used to cleave GAGs from cells bearing proteoglycans or glycoproteins on the cell surface. The cells from which GAGs can be harvested include prokaryotic and eukaryotic cells. One or more GAGs can be produced by any combination of methods provided herein and known in the art. Preferably, the one or more GAGs are a population of GAGs that is a structurally specific population of GAGs.

In some embodiments the GAG-modulating agent is produced by a cell. These cells can in some embodiments be co-cultured with the stem cells. These cells can be cells of any type, such as, mammalian cells, bacterial cells or genetically engineered cells. These cells include cells that have been altered (e.g., genetically) to express one or more GAG-modulating agents or to have altered expression of one or more GAG-modulating agents. Cells that have “altered expression” of one or more GAG-modulating agents include those that have expression of one or more GAG-modulating agents that is increased or decreased relative to the expression prior to the alteration. This includes expression of one or more GAG-modulating agents that is altogether eliminated or is introduced to a cell that previous to the alteration did not exhibit any expression of the one or more GAG-modulating agents. The cells can be altered by genetic and recombinant means that are well known in the art. For instance, the cell can be transfected with a vector that allows for the production and, preferably, the secretion of one or more GAG-modulating agents. Cells, for example, also can be transfected with a vector used to produce RNA transcripts for the purpose of reducing or eliminating the expression of one or more GAG-modulating agents. These and other methods will be well-known to those of ordinary skill in the art. In one embodiment, it is the stem cell itself that can be altered to express or have altered expression of one or more GAG-modulating agents.

The GAG-modulating agents or cells that expresses the GAG-modulating agents can be used to inhibit or promote stem cell differentiation in vivo or in vitro. In one embodiment stem cell differentiation to endothelial cells is promoted. In another embodiment stem cell differentiation to endothelial cells is inhibited. In still another embodiment stem cell differentiation is inhibited such that a cell population impoverished of endothelial cells is produced. “A population of cells that is impoverished of endothelial cells” includes any population of cells that is produced where endothelial cells are in the minority (of the whole population of cells) or are nonexistent. Therefore, such a cell population can have less than 50%, 40%, 30%, 25%, 20%, 15%, 10%, 5%, 2%, 1% or fewer endothelial cells. In another embodiment stem cell differentiation is inhibited such that a cell population is obtained that is enriched in muscle, neural or blood cells. If a population of cells is enriched in one type of cell, the cell population produced has a greater amount of that particular cell type as compared to the other cells. In some embodiments the enriched cells can represent 40%, 50%, 60%, 70%, 80%, 85%, 90%, 95%, 97%, 99% or greater of the total cell population. As compared to each of the other cell types, the enriched cell type will be in a greater amount. In still other embodiment stem cell differentiation is promoted such that a cell population that is primarily endothelial cells is produced. “A cell population that is primarily endothelial cells” is a population whereby greater than 50%, 60%, 70%, 80%, 85%, 90%, 95%, 97%, 99% or more of the cells are endothelial cells. The stem cell differentiation can, therefore, be modulated such that specific cell populations can be produced. The cells produced can be any kind of cells, such as mammalian cells (e.g., human cells).

Therefore, a method is provided to produce a population of cells, such as those described herein by contacting the microenvironment of a stem cell to inhibit or promote cell differentiation and obtaining a population of cells. The method can be in vitro or in vivo. In one embodiment the method takes place in culture. In another embodiment the method takes place in a subject. Compositions of these cell populations are also provided as are tissues that are engineered with these cell populations. In one embodiment, the obtained cell population is mammalian. In another embodiment, the obtained cell population is human. In still another embodiment, the obtained cell population is a therapeutic. In one embodiment, the resulting cell population is used in tissue engineering. In another embodiment, the obtained cell population or the engineered tissue is used in a subject to treat a disease state.

Two or three dimensional structures are any support onto which a GAG-modulating agent or a cell that expresses the GAG-modulating agent can be covalently or non-covalently bound. These structures include, for example, medical devices that can be implantable. The structure can be, for example, a scaffold, matrix, stent, shunt, valve, pacemaker, pulse generator, cardiac defibrillator, spinal stimulator, brain stimulator, sacral nerve stimulator, lead, inducer, sensor, screw, anchor, pin, adhesion sheet, needle, lens, joint, prosthetic/orthopedic implant, catheter, tube (e.g., tubes for lines and drains), suture, etc.

The stem cells of the methods and compositions provided herein can be any stem cell. Stem cells are intended to refer to non-differentiating stem cells and stem cells that may be undergoing differentiation. In one embodiment, the stem cells are totipotent, pluripotent, hematopoietic, mesenchymal, neural or progenitor stem cells. The stem cells can be mammalian, and in some embodiments the stem cells are human. In another embodiment the stem cell is an embryonic stem (ES) cell.

ES cells are pluripotent cells derived from the inner cell mass of developing blastocysts and have the unique ability to differentiate into any adult cell type (Cowan, C. A. et al. (2004) N Engl J Med 350, 1353-6). ES cells are easy to maintain in vitro in an undifferentiated pluripotent state and possess the potential to differentiate into any cell type. ES cells have been isolated from various mammalian sources including mice, non-human primates and recently also from humans (Thomson et al, 1998, Science, 282:1145-1147; Reubinoff et al, 2000, Nat Biotechnol, 18:399-404). These cells have the capacity of self-renewal, and they are also able to differentiate to generate various cell lineages when cultured under appropriate conditions, such as hematopoietic (Keller et al, Mol Cell Biol, 1993, 13:473-486; Pacacios et al, Proc Natl Acad Sci USA, 1995, 92:7530-7534), muscle (Rohwedel et al, Dev Biol, 1994, 164:87-101; Robbins et al, J Biol Chem, 1990, 265: 11905-11909), neuronal (Bain et al, Dev Biol 1995, 168:342-357) and endothelial (Risau et al, Development, 1988, 102:471-478). It has also been demonstrated that ES cell derived neuronal, cartilage, liver or endothelial tissue maintain their viability and continue to express proteins specific to the differentiated structures when they are injected into mice (Levenberg et al, Proc Natl Acad Sci USA, 2003, 100: 12741-46; Hara et al, Brain Res., 2004, 999(2):216-21; Yamamato et al, Hepatology, 2003, 37(5): 983-93; Meyer et al Brain Res., 2004, 1014: 131-44; Levenberg et al, Proc Natl Acad Sci USA, 2002, 99(7): 4391-6). Endothelial cells derived from ES cells express most known endothelial cell markers including VEGFR-2, Tie-2, vWF and VE-cadherin (Vittet et al, Blood, 1996, 88:3424-31), they can form capillary like structures in vitro, and can form microvessels and vasculature when transplanted into mice (Marchetti et al, J Cell Sci., 2002, 115:2075-85; Kaufman et al, Blood, 2004, 103(4): 1325-32). Thus ES cells, with their self renewal and pluripotency, suggest an unlimited source of cells that can generate all types of tissues and therefore provide valuable sources for tissue transplantation, regeneration and engineering.

Methods and compositions whereby stem cell differentiation is promoted or inhibited in a subject are provided. Therefore, the methods and compositions provided herein can be used for a variety of treatment endpoints. In one aspect of the invention methods of treating a subject by the administration of one or more GAG-modulating agents or one or more cells that express one or more GAG-modulating agents is provided in an effective amount to treat the subject. In some embodiments the GAG-modulating agent is administered in an amount effective to modulate stem cell differentiation to endothelial cells. “An amount effective to modulate stem cell differentiation to endothelial cells” refers to any amount of a GAG-modulating agent that alone or in combination with another agent is able to inhibit or promote stem cell differentiation to endothelial cells. To “inhibit” is to reduce or eliminate stem cell differentiation to endothelial cells, while to “promote” is to cause stem cell differentiation to endothelial cells. Methods of treating a subject can also be accomplished using the cell populations and engineered tissues produced by the methods provided herein.

The methods and compositions can be useful for regenerative medicine. One of the attractive targets for regenerative medicine is the establishment of a viable vasculature. The vascular system is laid down during early developmental stages, and is fairly quiescent in the adult. Neovascularization (the formation of new blood vessels) can also occur in the adult, albeit in a strictly regulated manner in certain physiological conditions, such as the reproductive cycle, tissue regeneration and wound healing. In contrast, some pathological conditions, such as diabetes, hypercholesterolemia and advanced age, are associated with impaired neovascularization which results in ischemic tissue. This impaired neovascularization is in large part due to endothelial cell dysfunction and the promotion of neovascularization in such conditions has significant therapeutic implications in treating patients. One way to achieve this is via engineering of the impaired vasculature component in vivo, or the ex-vivo regeneration and transplantation of reconstituted vascular tissue. The essential component of the vasculature that plays a role in neovascularization is the endothelial cells lining the vessels. Other studies have attempted to regenerate the vascular tissue by using primary endothelial cells and endothelial progenitor cells (Joyce, N. C., and Zhu, C. C. (2004) Cornea 23, S8-S19 and Murasawa, S. (2004) Nippon Ronen Igakkai Zasshi 41, 48-50). However, these approaches are limited by the finite life span of primary cells, and by the low abundance of progenitor cells in circulating blood.

Provided herein, therefore, are methods and compositions to treat a subject in need of vascularization (blood vessel formation or neovascularization). Such subjects include, but are not limited to, those with a chronic wound, those in need of the restoration of cardiac function, those with ischemic tissue as well as those that have or are at risk of having coronary artery disease, diabetes, hypercholesterolemia, etc. Such subjects also include those that are of an advanced age. In some embodiments a subject that is of an advanced age is one that is greater than 65, 70, 80, 85, 90 or 95 years old. It has now been found that GAG-modulating agents can be used to promote the stem cell differentiation to endothelial cells. Therefore, the GAG-modulating agents or cells that expresses the GAG-modulating agents provided can be administered to promote stem cell differentiation to endothelial cells in vivo. Additionally, the compositions of cell populations and engineered tissues thereby produced as provided herein can also be used to treat a subject in need of blood vessel formation (e.g., neovascularization). Examples of diseases that can be treated by stimulation of stem cell differentiation to endothelial cells, or by use of an endothelial cell population or engineered tissue include, but are not restricted to, conditions in which an ischemic tissue is formed in a subject as a result of impaired blood vessel formation. These conditions include diseases, such as diabetes, stroke, angina, CAD, hypercholesterolemia and advanced age. Chronic wounds constitute another example of a disease state where blood vessel formation may be desirable.

Under some pathophysiological conditions, blood vessel formation takes place in an unregulated, persistent manner (e.g. tumor neovascularization, arthritis). For these conditions, methods to inhibit blood vessel formation carries remarkable therapeutic implications. Since endothelial cells are essential components of blood vessels, it is possible to regulate blood vessel formation through regulating endothelial cell generation. In mammals, generation of endothelial cells occurs by two mechanisms; through proliferation of pre-existing endothelial cells and through differentiation of progenitor stem cells. Other groups have disclosed methods to inhibit the formation of blood vessels through inhibition of proliferation of pre-existing endothelial cells (e.g., U.S. Pat. Nos. 6,743,428; 6,703,049; 6,683,051; 5,268,384; 5,001,116). However, for an effective therapeutic outcome, it is not sufficient only to inhibit proliferation of pre-existing endothelial cells; it is important to develop methods to inhibit differentiation of progenitor stem cells into endothelial cells. For such pathophysiological cases, the methods and compositions provided can be applied to the inhibition of progenitor stem cell differentiation to endothelial cells to obtain the desired therapeutic outcomes.

Therefore, methods and compositions are provided for use in treating a subject with undesired blood vessel formation. Therefore, the compositions and methods provided can result in the reduction or elimination of stem cell differentiation to endothelial cells. The methods and compositions, therefore, can be used to treat a variety of pathological conditions such as cancer (i.e., tumor angiogenesis) and arthritis, through the inhibition of blood vessel formation. This is accomplished, for example, through the use of GAG-modulating agents that inhibit stem cell differentiation to endothelial cells.

Proliferation of endothelial and vascular smooth muscle cells is the main feature of neovascularization. Thus the substrates of the invention are useful for preventing proliferation and, therefore, inhibiting or arresting altogether the progression of the angiogenic condition which depends in whole or in part upon such neovascularization. The compositions and methods provided may be used, for instance, in a method for inhibiting angiogenesis. “Angiogenesis” often occurs in tumors when endothelial cells secrete a group of growth factors that are mitogenic for endothelium causing the elongation and proliferation of endothelial cells which results in a generation of new blood vessels. Neovascularization, or angiogenesis, is the growth and development of new arteries. It is critical to the normal development of the vascular system, including injury-repair. There are, however, conditions characterized by abnormal neovascularization, including diabetic retinopathy, neovascular glaucoma, rheumatoid arthritis, and certain cancers. For example, diabetic retinopathy is a leading cause of blindness. There are two types of diabetic retinopathy, simple and proliferative. Proliferative retinopathy is characterized by neovascularization and scarring. About one-half of those patients with proliferative retinopathy progress to blindness within about five years. As used herein, an angiogenic condition means a disease or undesirable medical condition having a pathology including neovascularization. Cancer angiogenic conditions are solid tumors and cancers or tumors otherwise associated with neovascularization such as hemangioendotheliomas, hemangiomas and Kaposi's sarcoma.

Other examples of cancers, include melanoma, hepatic adenocarcinoma, prostatic adenocarcinoma or osteosarcoma. Other cancers include biliary tract cancer; bladder cancer; breast cancer; brain cancer including glioblastomas and medulloblastomas; Burkitt's lymphoma, cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease; lung cancer including small cell lung cancer and non-small cell lung cancer; lymphomas including Hodgkin's disease and lymphocytic lymphomas; neuroblastomas; oral cancer including squamous cell carcinoma; esophageal cancer; ovarian cancer including those arising from epithelial cells, stromal cells, germ cells and mesenchymal cells; pancreatic cancer; rectal cancer; sarcomas including leiomyosarcoma, rhabdomyosarcoma, liposarcoma, fibrosarcoma, and synovial sarcoma; skin cancer including Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

The methods and compositions provided can be used in the treatment of subjects having or at risk of having any of the conditions provided herein or otherwise apparent due to the teachings provided.

The promotion of angiogenesis or neovascularization, however, can also be desirable. For example, angiogenesis would be desirable in tissue engineering applications, such as with the use of stents, prosthetic implants, skin grafts, artificial skin, vascular grafts, or any application where increased vascularization is desirable. Compositions and methods are, therefore, provided for the promotion of angiogenesis, preferably, for tissue engineering applications. In one compositions and methods provided can also include an angiogenic factor such as VEGF, FGF, EGF, PDGF or hepatocyte growth factor (HGF).

The compositions and methods provided can also be used in the treatment of disorders associated with coagulation. A “disease associated with coagulation” as used herein refers to a condition characterized by inflammation resulting from an interruption in the blood supply to a tissue, which may occur due to a blockage of the blood vessel responsible for supplying blood to the tissue such as is seen for myocardial, cerebral infarction, or peripheral vascular disease, or as a result of embolism formation associated with conditions such as atrial fibrillation or deep venous thrombosis. A cerebral ischemic attack or cerebral ischemia is a form of ischemic condition in which the blood supply to the brain is blocked. This interruption in the blood supply to the brain may result from a variety of causes, including an intrinsic blockage or occlusion of the blood vessel itself, a remotely originated source of occlusion, decreased perfusion pressure or increased blood viscosity resulting in inadequate cerebral blood flow, or a ruptured blood vessel in the subarachnoid space or intracerebral tissue. Coagulation associated diseases/states also include disseminated intravascular coagulation, venous stasis, pregnancy, cancer, hemophilia, clotting factor deficiencies, etc.

The invention also contemplates the treatment of subjects having or at risk of developing a neurodegenerative disorder, such as a neurodegenerative disease or suffering an injury to nerve cells. Neuronal cells are predominantly categorized based on their local/regional synaptic connections (e.g., local circuit interneurons vs. longrange projection neurons) and receptor sets, and associated second messenger systems. Neuronal cells include both central nervous system (CNS) neurons and peripheral nervous system (PNS) neurons. There are many different neuronal cell types. Examples include, but are not limited to, sensory and sympathetic neurons, cholinergic neurons, dorsal root ganglion neurons, proprioceptive neurons (in the trigeminal mesencephalic nucleus), ciliary ganglion neurons (in the parasympathetic nervous system), etc. A person of ordinary skill in the art will be able to easily identify neuronal cells and distinguish them from non-neuronal cells such as glial cells, typically utilizing cell-morphological characteristics, expression of cell-specific markers, secretion of certain molecules, etc.

“Neurodegenerative disorder” is defined herein as a disorder in which progressive loss of neurons occurs either in the peripheral nervous system or in the central nervous system. Examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapic, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofliscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor). and Wernicke-Korsakoff's related dementia. Neurodegenerative diseases affecting sensory neurons include Friedreich's ataxia, diabetes, peripheral neuropathy, and retinal neuronal degeneration. Neurodegenerative diseases of limbic and cortical systems include cerebral amyloidosis, Pick's atrophy, and Retts syndrome. The foregoing examples are not meant to be comprehensive but serve merely as an illustration of the term “neurodegenerative disorder.”

The terms “treat” and “treating” as used herein refer to reducing or eliminating the symptoms of the disease or condition, improving the health of the subject in some way, reversing the progression of a disease or condition or altogether eliminating the disease or condition. Such terms are also intended to include the reduction the possibility of the subject from developing the disease or condition. When the disease or condition is cancer, “treat” or “treating” refers to inhibiting completely or partially the proliferation or metastasis of a cancer or tumor cell, as well as inhibiting any increase in the proliferation or metastasis of a cancer or tumor cell. Treat or treating also refers to retarding the proliferation or metastasis of tumor cells in a subject. Additionally, treat or treating may include the elimination or reduction of the symptoms associated with the tumor cell proliferation or metastasis. A “subject having a cancer” is a subject that has detectable cancerous cells. The cancer may be a malignant or non-malignant cancer.

A “subject at risk” is a subject that has a high probability of developing a certain disease or disorder. As an example, “a subject at risk of having a cancer” is a subject who has a high probability of developing cancer. Subjects at risk include, for instance, subjects having a genetic abnormality, the presence of which has been demonstrated to have a correlative relation to a higher likelihood of developing the disease or condition, subjects exposed to agents or have a lifestyle associated with the disease or condition, or subjects who have previously been treated for the disease or condition. A subject at risk can be treated with the compositions and methods provided, alone or in combination with an additional therapeutic.

Methods and compositions related to the inhibition of stem cell differentiation to endothelial cells in order to produce a population of blood cells, neural cells and/or muscle cells is also provided. In one embodiment such a population is an enriched population of blood cells, neural cells or muscle cells. In another embodiment such a population of cells has at least one type of cell (blood, neural or muscle) that is in greater amounts than the endothelial cells of the population. Such cell populations can have a variety of therapeutic applications, which include therapeutic applications in which blood cells are generated from stem cells for purposes, such as blood transfusion. Other conditions include those in which neural cells are generated from stem cells for treatment of neurodegenerative diseases, and the method of obtaining neural cells from stem cells involves the inhibition of differentiation of stem cells to endothelial cells. The methods and compositions provided herein, therefore, can be used to treat neurodegenerative disorders or nervous system injury.

The methods and compositions herein can be combined with the administration of an additional therapeutic agent.

Additional therapeutic agents include anti-cancer agents. Anti-cancer agents include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Additional agents further include agents that treat the side-effects of radiation therapy, such as anti-emetics, radiation protectants, etc.

Anti-cancer agents also can include cytotoxic agents and agents that act on tumor neovasculature. Cytotoxic agents include cytotoxic radionuclides, chemical toxins and protein toxins. The cytotoxic radionuclide or radiotherapeutic isotope preferably is an alpha-emitting isotope such as ²²⁵Ac, ²At, ²¹²Bi, ²¹³Bi, ²¹²Pb, ²²⁴Ra or ²²³Ra. Alternatively, the cytotoxic radionuclide may a beta-emitting isotope such as 186Rh, 188Rh, ¹⁷⁷Lu, 90y, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes ¹²⁵I, ¹²³I, or ⁷⁷Br.

Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Toxins also include poisonous lectins, plant toxins such as ricin, abrin, modeccin, botulina and diphtheria toxins. Of course, combinations of the various toxins are also provided thereby accommodating variable cytotoxicity. Other chemotherapeutic agents are known to those skilled in the art.

Agents that act on the tumor vasculature can include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein), interferon inducible protein 10 (U.S. Pat. No. 5,994,292), and the like. Anticancer agents also include immunomodulators such as α-interferon, γ-interferon, and tumor necrosis factor alpha (TNFα).

The compositions and methods provided herein can be combined with other therapeutic agents used to promote nerve regeneration or treat neurodegenerative disease.

For example, antiparkinsonian agents include but are not limited to Benztropine Mesylate; Biperiden; Biperiden Hydrochloride; Biperiden Lactate; Carmantadine; Ciladopa Hydrochloride; Dopamantine; Ethopropazine Hydrochloride; Lazabemide; Levodopa; Lometraline Hydrochloride; Mofegiline Hydrochloride; Naxagolide Hydrochloride; Pareptide Sulfate; Procyclidine Hydrochloride; Quinelorane Hydrochloride; Ropinirole Hydrochloride; Selegiline Hydrochloride; Tolcapone; Trihexyphenidyl Hydrochloride. Drugs for the treatment of amyotrophic lateral sclerosis include but are not limited to Riluzole. Drugs for the treatment of Paget's disease include but are not limited to Tiludronate Disodium.

Examples of additional therapeutics also include anticoagulation agents, antiplatelet agents and thrombolytic agents.

Anticoagulants include, but are not limited to, heparin, modified heparins, dermatan sulfate, oversulfated dermatan sulfate, warfarin, coumadin, dicumarol, phenprocoumon, acenocoumarol, ethyl biscoumacetate, and indandione derivatives.

Antiplatelet agents include, but are not limited to, aspirin, thienopyridine derivatives such as ticlopodine and clopidogrel, dipyridamole and sulfinpyrazone, as well as RGD mimetics and also antithrombin agents such as, but not limited to, hirudin.

Thrombolytic agents include, but are not limited to, plasminogen, a₂-antiplasmin, streptokinase, antistreplase, tissue plasminogen activator (tPA), and urokinase.

Additional agents for the inhibition of coagulation include clotting factors and antithrombins, such as antithrombin 3.

Similarly, as the compositions and methods provided can promote wound healing additional therapeutics also include, collagen to increase wound strength and promote platelet aggregation and fibrin formation; growth factors, such as platelet-derived growth factor, platelet factor 4, transforming growth factor-s; tissue factor VIIa, thrombin, fibrin, plasminogen-activator initiator, adenosine diphosphate, etc.

Additionally, anti-inflammatory agents can also be used and are included as additional therapeutics. Anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexarnethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

The methods provided herein can further comprise the step of assessing stem cell differentiation to endothelial cells. As used herein, “assessing stem cell differentiation to endothelial cells” refers to determining whether stem cell differentiation to endothelial cells is inhibited or promoted. In order to assess stem cell differentiation to endothelial cells any of a number of molecules can be analyzed. For example, the expression of markers that are specific for the stem cells and/or markers that are specific for endothelial cells can be determined. Stem cell specific markers include Oct-4. Endothelial cell markers include wVf, VEGF-R2, VE-cadherin, eNOS and Tie-2. The expression of such markers provides an indication of stem cell differentiation and can be detected with any molecules that bind to the markers or to nucleic acids that encode the markers. Stem cell differentiation can also be assessed by measuring MAPK factors, such as ERK. As an example, the phosphorylation of ERK can be determined. In a further embodiment the expression of one or more markers is determined. Methods for determining the expression of markers are known to those of skill in the art and are also provided herein in the Examples.

Effective amounts of the therapeutic agents provided are administered to subjects in need of such treatment. Effective amounts are those amounts which will result in the desired therapeutic endpoint, such as the reduction in cellular proliferation or metastasis, the promotion or inhibition of neural regeneration, the inhibition or promotion of stem cell differentiation to an endothelial cell population etc., without causing other medically unacceptable side effects. Such amounts can be determined with no more than routine experimentation. Effective amounts can mean that one therapeutic is administered in an amount effective to reach a desirable therapeutic endpoint or it can mean that a combination of therapeutic agents is necessary to reach the desirable therapeutic endpoint. It is believed that doses ranging from 1 nanogram/kilogram to 100 milligrams/kilogram, depending upon the mode of administration, will be effective. The absolute amount will depend upon a variety of factors (including whether the administration is in conjunction with other methods of treatment, the number of doses and individual patient parameters including age, physical condition, size and weight) and can be determined with routine experimentation. It is preferred generally that a maximum dose be used, that is, the highest safe dose according to sound medical judgment.

A subject is any human or non-human vertebrate, e.g., dog, cat, horse, cow, pig. In another embodiment the subject is one otherwise not in need of the compositions and methods of treatment as provided herein. Such a subject is one that would not receive the compositions and treatments provided except for the need for the modulation of stem cell differentiation to endothelial cells as provided herein. In one embodiment the need is for the promotion of stem cell differentiation to endothelial cells. In another embodiment the need is for the inhibition of stem cell differentiation to endothelial cells.

Kits comprising the surfaces and compositions discussed herein are also provided. The kits can further include diagnostic agents, such as labels or an additional therapeutic agent.

In general, when administered for therapeutic purposes, the medical devices of the invention are applied in pharmaceutically acceptable form.

In other embodiments the medical devices/substrates provided are sterile.

In general, when administered for therapeutic purposes, the formulations of the invention are applied in pharmaceutically acceptable solutions. Such preparations may routinely contain pharmaceutically acceptable concentrations of salt, buffering agents, preservatives, compatible carriers, adjuvants, and optionally other therapeutic ingredients.

The compositions of the invention may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% WNV); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The present invention provides pharmaceutical compositions, for medical use, with one or more pharmaceutically acceptable carriers and optionally other therapeutic ingredients. The term “pharmaceutically-acceptable carrier” as used herein, and described more fully below, means one or more compatible solid or liquid filler, dilutants or encapsulating substances which are suitable for administration to a human or other animal. In the present invention, the term “carrier” denotes an organic or inorganic ingredient, natural or synthetic, with which the active ingredient is combined to facilitate the application. The components of the pharmaceutical compositions also are capable of being commingled with each other, in a manner such that there is no interaction which would substantially impair the desired pharmaceutical efficiency.

A variety of administration routes are available. The particular mode selected will depend, of course, upon the particular active agent selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. A preferred mode of administration is a parenteral route. The term “parenteral” includes subcutaneous injections, intravenous, intramuscular, intraperitoneal, intrasternal injection or infusion techniques. Other modes of administration include systemic, local, topical, site-specific, oral, mucosal, rectal, vaginal, sublingual, intranasal, intratracheal, inhalation, ocular, transdermal, etc. Other modes include the implantation or transplantation of structure such as a medical device. In some embodiment the implantation or transplantation is site-specific (or localized to the site where a therapeutic effect would be beneficial). In some embodiments the compositions provided are administered to a joint. In other embodiment the compositions provided are targeted to an area in need of blood vessel formation or blood vessel formation inhibition.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The compositions may conveniently be presented in unit dosage form and may be prepared by any of the methods well known in the art of pharmacy.

Other delivery systems can include time-release, delayed release or sustained release delivery systems. Such systems can avoid repeated administrations of the compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. No. 4,452,775 (Kent); U.S. Pat. No. 4,667,014 (Nestor et al.); and U.S. Pat. Nos. 4,748,034 and 5,239,660 (Leonard) and (b) diffusional systems in which an active component permeates at a controlled rate through a polymer, found in U.S. Pat. No. 3,832,253 (Higuchi et al.) and U.S. Pat. No. 3,854,480 (Zaffaroni). In addition, a pump-based hardware delivery system can be used, some of which are adapted for implantation.

Controlled release can also be achieved with appropriate excipient materials that are biocompatible and biodegradable. These polymeric materials which effect slow release may be any suitable polymeric material for generating particles, including, but not limited to, nonbioerodable/non-biodegradable and bioerodable/biodegradable polymers. Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate.

Examples of preferred non-biodegradable polymers include ethylene vinyl acetate, poly(meth)acrylic acid, polyamides, copolymers and mixtures thereof.

Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

The directed differentiation of ES cells holds a potential for regenerative medicine; therefore, an understanding of the mechanisms regulating self-renewal and cell fate decisions is helpful. Previously, attempts have been made to elucidate the key regulatory components of differentiation using transcriptomic and proteomic approaches; however, these studies have failed to capture the complete complexity of this process. Glycosaminoglycans, e.g., HSGAGs, are components of the extracellular matrix and constitute one of the major components of a cell's glycome. Murine ES cells, directed to differentiate under LIF-free conditions, progressively lost the stem cell marker, Oct-4, and acquired endothelial cells markers, such as von Willebrand factor, VE-cadherin, VEGF-R2 and eNOS, as detected by flow cytometry, confocal microscopy and real-time PCR. Compositional analysis of HSGAG structure by capillary electrophoresis revealed an increase in the quantity of HSGAGs with progressive differentiation, which was paralleled by an increase in the transcript levels of key HSGAG biosynthetic enzymes. Ablation of the HSGAG biosynthetic machinery through sodium chlorate treatment, or the enzymatic decomposition of HSGAGs via treatment with heparinases, inhibited the formation of endothelial cells, although differentiation to other cell types did proceed as evidenced from the progressive loss of the Oct-4 signal. Reconstitution of the HSGAG moiety in sodium chlorate treated cells by the exogenous addition of heparin partially recovered the formation of endothelial cells, suggesting that HSGAGs play a role in the differentiation of ES cells into endothelial cells. Western blot analysis of the phospho-ERK levels suggest that HSGAGs impinge on differentiation of ES cells into endothelial cells possibly through the MAPK pathway. Therefore, the role of the glycome is implicated in the directed differentiation of stem cells.

Materials and Methods

Cell Culture

Mouse embryonic stem cells (J1) (gift from Bevin Engelward, Massachusetts Institute of Technology, Cambridge, Mass.) were grown in DMEM with 2 mM L-glutamine (GIBCO, Carlsbad, Calif.) adjusted to contain 100 mM sodium pyruvate, 10 mM nonessential amino acids and 1.5 g/L sodium bicarbonate, with 15% fetal bovine serum (Hyclone, South Logan, Utah) and supplemented with 100 units/mL penicillin G (Sigma, St. Louis, Mo.), 100 μg/mL streptomycin sulfate (Sigma), 30 mM beta-mercaptoethanol (Sigma) and 1000 units/ml murine leukemia inhibitory factor, LIF (Chemicon, Temecula, Calif.). The cells were plated in 10 cm² cell culture dishes, coated with 0.1% gelatin B (Sigma) and grown at 37° C. in a 5% CO₂ humidifier incubator. The culture medium was changed every 2 days. The cells were subcultured at a split ratio of 1:10 when they reached 80% to 90% confluency, using a 0.25% trypsin-EDTA solution (Sigma). To induce formation of embryoid bodies and differentiation into endothelial cells, J1 cells were plated at a density of 1.25×10⁵ cells/100 mm dish, or 3×10⁴ cells/6 wells and cultured in the absence of LIF. Fresh media was replenished every 2 days.

Enzymatic and Chemical Treatment of Cells

Heparinase III (Hep-III) was prepared as described previously (Godavarti, R., et al. (1996) Biochemistry 35, 6846-52). Heparinase I (Hep-I) was a generous gift of Momenta Pharmaceuticals (Cambridge, Mass.). Sodium chlorate and heparin were purchased from Sigma and Cambrex BioWhittaker (East Rutherford, N.J.), respectively. To enzymatically modify the HSGAG glycome signature of the cells, cells were washed with phosphate buffer saline (PBS) and incubated with Hep-I or Hep-III for 30 minutes every day at 37° C. in serum free DMEM. Digested HSGAG residues were removed by washing the cells, and cells were then fed with fresh media. In an additional experiment, cells were cultured with medium containing sodium chlorate, which blocks the sulfation of HSGAGs. In a reversal experiment, heparin was added to the culture to overcome the chlorate induced synthetic block. Treated cells were analyzed for differentiation into endothelial cells between day 3 and day 15. Hep-III was added to the cells at a concentration of 2.5 μg/mL, Hep-I was added at a concentration of 1.5 μg/mL, sodium chlorate at 10 mM and heparin (20 μg). The concentrations of enzymes were optimized to exhaustively cleave all cell surface HSGAGs in the given period of time. All enzymes and chemicals were diluted in serum free DMEM.

Flow Cytometry

Cells were harvested at various time points during differentiation, incubated with rat monoclonal antibody against CD16/CD32 (1:50 dilution, Pharmingen, San Diego, Calif.) to block Fcγ receptors and incubated further with a primary antibody against von Willebrand factor (vWF) (Dako (Carpinteria, Calif.), rabbit polyclonal, added at a 1:100 dilution). An isotype-matched polyclonal antibody IgG (Pharmingen) was used as the control. Cells were then washed twice and were incubated with FITC-conjugated secondary antibody (Jackson ImmunoResearch, West Grove, Pa.) in a 1:100 dilution, washed twice and taken up in OptiMEM (GIBCO/BRL, Carlsbad, Calif.) and analyzed on a Becton Dickinson (Franklin Lakes, N.J.) FACScan flow cytometer (excitation 488 nm, argon laser; emission 580/30).

Confocal Microscopy

For microscopic analysis of endothelial cells, the differentiating J1 cells were fixed in cold methanol at designated time points, blocked in goat serum and probed overnight with a rabbit primary antibody against vWF, an endothelial cell marker, or Oct-4, a stem cell marker. The sections were washed and re-probed with a goat secondary antibody coupled to FITC. The nuclei were counterstained with propidium iodide. Images were captured using a Leica LSM510 confocal microscope at a 512×512 pixels resolution (Leica, Bannockburn, Ill.). Fluorochromes were excited with 488 nm and 543 nm laser lines, and the images were captured using 505-530 BP and 565-615 BP filters at a 512×512 pixels resolution.

Real-Time PCR

RNA was isolated from J1 cells using TRIzol (Invitrogen, Carlsbad, Calif.) and RNAlater (Qiagen, Valencia, Calif.) according to the manufacturer's protocol. Single-stranded cDNA was generated via oligo-dT primed reverse transcription, and genes serving as markers for differentiation were quantified using an Abi Prism 7700 real-time PCR thermocycler (Applied Biosystems, Foster City, Calif.). Expression levels were obtained for the marker genes Oct-4, VE-cadherin, VEGF-R2, and eNOS, with β-actin used as a control. PCR conditions involved denaturation for 10 min at 95° C., followed by 40 cycles of denaturation for 20 sec at 94° C., and annealing and extension for 1 min at 60° C. cDNA isolated from J1 cells and primers for the described genes were mixed with SYBR Green PCR Mastermix (Applied Biosystems) for real-time quantification according to the manufacturers instructions. Primers used were 5′-ccaatcagcttgggctagag-3′ (SEQ ID NO: 1) and 5′-ctgggaaaggtgtccctgta-3′ (SEQ ID NO: 2) for Oct-4; 5′-accgagagaaacaggctgaa-3′ (SEQ ID NO: 3) and 5′-agacggggaagttgtcattg-3′ (SEQ ID NO: 4) for VE-cadherin; 5′-ggacagtgctccaaccaaat-3′ (SEQ ID NO: 5) and 5′-gttcacactgcagacccaga-3′ (SEQ ID NO: 6) for TEE-2; 5′-gctttcggtagtgggatgaa-3′ (SEQ ID NO: 7) and 5′-ggccttccatttctgtacca-3′ (SEQ ID NO: 8) for VEGF-R2; 5′-tcttcgttcagccatcacag-3′ (SEQ ID NO: 9) and 5′-cctatagcccgcatagcgta-3′ (SEQ ID NO: 10) for ENOS; 5‘-agccatgtacgtagccatcc-’3 (SEQ ID NO: 11) and 5‘-ctctcagctgtggtggtgaa-’3 (SEQ ID NO: 12) for β-actin. The sulfotransferase primers and conditions were chosen. Relative and normalized levels of gene expression were obtained according to the equation: 2^{-(Ct(gene)-Ct(β-actin))} where Ct is the cycle number at which amplification of each gene crossed an arbitrary threshold within the exponential phase of amplification. Gene expression levels were further normalized to expression levels prior to differentiation.

Isolation and Compositional Analysis of HSGAGs

Cell surface HSGAG fragments were isolated from J1 cells at various stages of differentiation. Briefly, cells were washed with PBS and treated with trypsin/EDTA (GibcoBRL) at 37° C. for 25 min to harvest cell surface proteoglycans. The resulting cell/trypsin solution was boiled for 10 min to deactivate the trypsin and other proteins. The solution was centrifuged at 4500 g, the supernatant was collected and concentrated by centrifuging in a Centriprep-3 (Amicon, Beverly, Mass.). The concentrated supernatant was run through ultrafree-DEAE (Pharmacia, Piscataway, N.J.) that had been equilibrated with 0.1M sodium phosphate buffer, pH6.0, that contained 0.15M NaCl. The bound HSGAG fragments were washed and eluted with 0.1M sodium phosphate buffer, pH6.0, that contained 1.0M NaCl. The fragments were then concentrated and buffer exchanged into ultra pure water by application to a Microcon filter (molecular weight cutoff=3,000Da). The samples were exhaustively digested overnight with a mixture of Hep-I and Hep-III (1 milliuniteach) in 25 mM sodium acetate and 1 mM calcium acetate, pH7.0. The samples were analyzed by capillary electrophoresis using a high-sensitivity flow cell under reverse polarity with a running buffer of 50 mM Tris/phosphate, pH2.5. Identities of the resultant saccharides were determined based on co-migration with known standards.

Western Blotting

For elucidating the biochemical pathways involved downstream of the modulation of the cell surface sugars, protein contents of the embryoid bodies were solubilized by rapid mixing with 3×SDS sample buffer under reducing conditions. Equivalent amounts of protein per sample were electrophoretically resolved on 4-12% gradient polyacrylamide gels and transferred onto a nitrocellulose (0.22 μm) membrane. The membrane was subsequently probed with a phosphor-ERK antibody (1:800 dilution, Cell Signaling Technologies, Danvers, Mass.), which specifically detects the phosphorylated forms of ERK1 and 2. The signal was amplified using a 1:2000 dilution of the appropriate horseradish peroxidase-conjugated secondary antibody (BioRad, Hercules, Calif.), and the immunocomplexes were visualized using enhanced chemiluminescence detection (Amersham Life Science, Piscataway, N.J.). The signal was normalized to the expression of total ERK1/2, which was detected on the same blot using ERK1/2 specific antibodies (Santa Cruz, Santa Cruz, Calif., used at a 1:200 dilution). Quantification of the luminescence signal was carried out by a Kodak 2000R imager.

Statistical Analysis

Statistical significance was tested using the Students t-test or one-way ANOVA followed by Dunnets or Friedman's Post-Hoc test (Graphpad Prism 3 software, Graphpad Software, San Diego, Calif.). P<0.05 was considered to be significant.

Results

ES Cells Differentiate Significantly Towards an Endothelial Cell Population

To optimize the conditions for efficient differentiation of ES cells into endothelial cells in vitro, the expression levels of ES cell specific and endothelial cell specific markers were analyzed at different stages of differentiation under different cell culture conditions. Specifically, the effects of cell density, the ECM content (i.e., gelatin, collagen, laminin, matrigel), exogenous addition of growth factors (i.e., VEGF, FGF, HGF and insulin) and different types of serum (i.e., Sigma fetal bovine serum (FBS) and Hyclone FBS) were tested. Conditions that gave a maximum of 30% differentiation into endothelial cells were achieved by plating the undifferentiated cells onto gelatin B coated tissue culture dishes, at a concentration of 1.25×10⁵ cells/100 mm dish, in the presence of 15% Hyclone FBS, 30 mM beta-mercaptoethanol, 1 mM sodium pyruvate, and in the absence of LIF without further passaging for 7-15 days. No effects of exogenous addition of growth factors were detected, possibly due to the presence of sufficient levels of growth factors either coming from the FBS or produced by the autocrine signaling of ES cells. The ES cell specific marker analyzed was Octamer-4, Oct-4, (Pesce, M., et al. (1998) Bioessays 20, 722-32), and the endothelial cell specific markers analyzed were von Willebrand factor (vWF) (Sadler, J. E. (1991) J Biol Chem 266, 22777-80), vascular endothelial growth factor receptor-2 (VEGF-R2) (Yamaguchi, T. P., et al. (1993) Development 118, 489-98), vascular endothelial cadherin (VE-cad) (Lampugnani, M. G., et al. (1992) J Cell Biol 118, 1511-22) and endothelial cell specific nitric oxide synthase (eNOS) (Alderton, W. K., et al. (2001) Biochem J 357, 593-615).

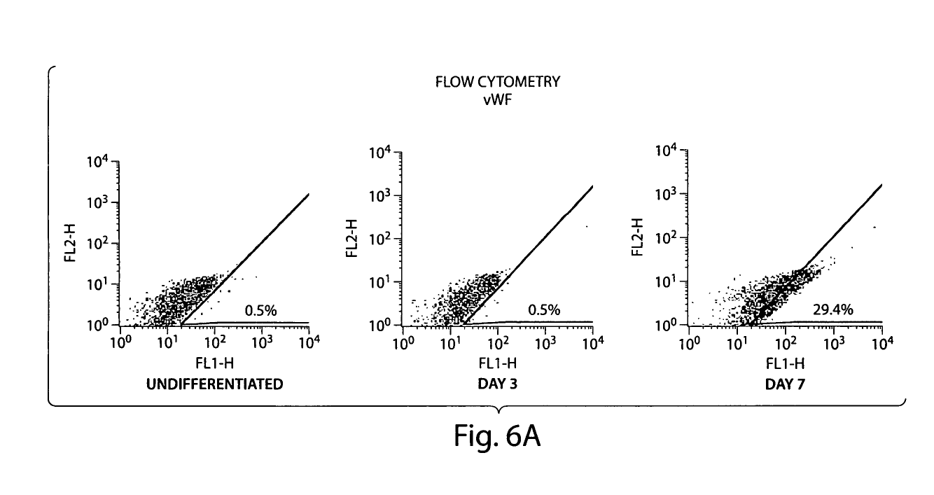

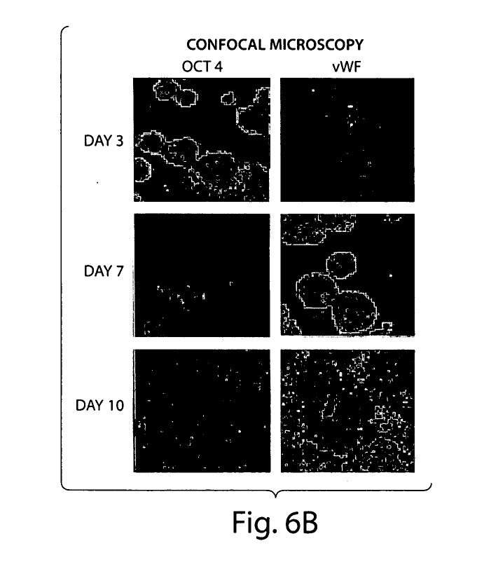

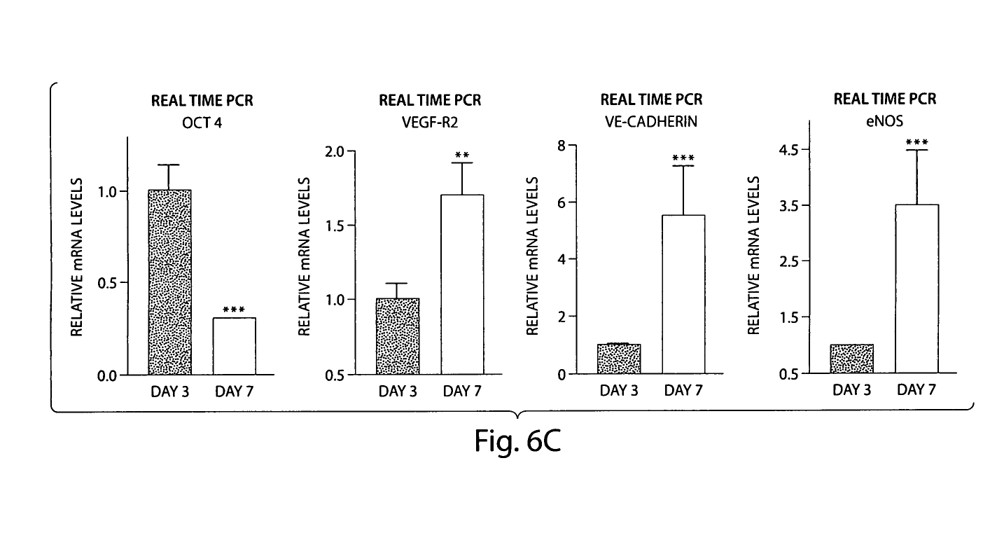

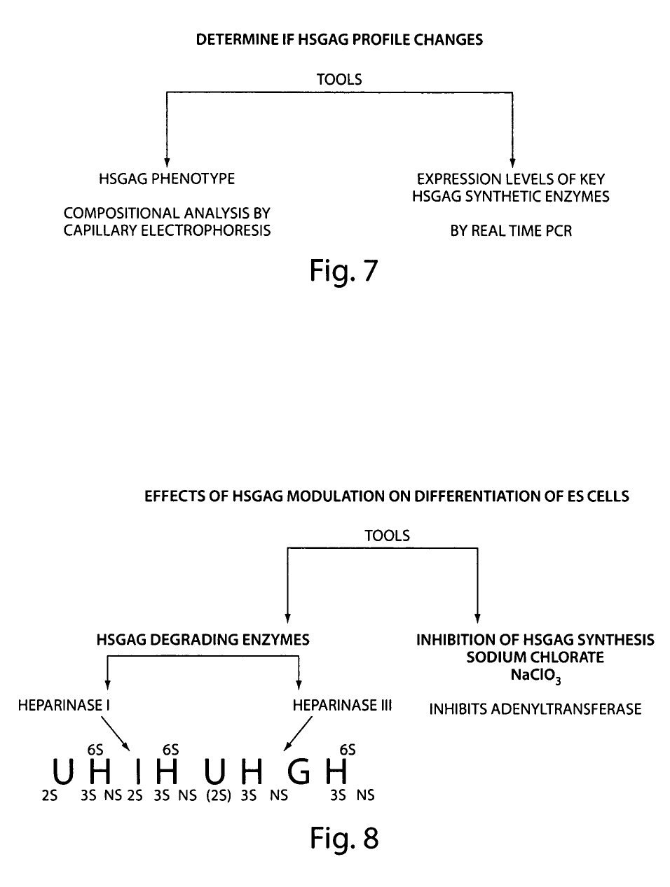

The flow-cytometry analysis of the expression of vWF in differentiating ES cells showed an increase in the proportion of cells expressing detectable levels of vWF over time (FIG. 6A). Similarly, confocal microscopy analysis of differentiating ES cells showed that expression levels of vWF protein increased during differentiation reaching a maximum between days 7-15 (FIG. 6B). Consistently, the expression levels of the ES cell marker, Oct-4, decreased progressively with time (FIG. 6B). This temporally converse expression of vWF and Oct-4 indicated an efficient differentiation of ES cells towards an endothelial cell population. Since vWF is also expressed in megakaryocytes, although to a lesser extent (Sadler, J. E. (1991) J Biol Chem 266, 22777-80), the results were confirmed by analyzing the temporal transcriptomal expression of other endothelial cell-specific markers, VEGF-R2, VE-cadherin and eNOS during differentiation. The real-time PCR results showed a decrease in the transcript levels of Oct-4, and an increase in the transcript signals of VEGF-R2, VE-cadherin and eNOS, 7 days after induction of differentiation (FIG. 6C). These results are consistent with the results from the confocal microscopy and flow-cytometry experiments and suggest significant differentiation towards an endothelial cell population.

HSGAG Synthesis is Upregulated During Differentiation of ES Cells

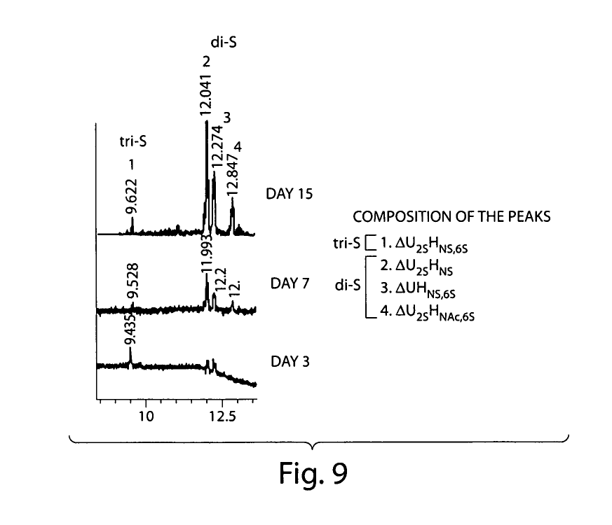

To investigate the role of HSGAGs in the differentiation of ES cells, the compositional changes in HSGAGs during differentiation were analyzed. For this purpose, cell surface HSGAGs were harvested at different stages of differentiation, normalized to cell number and subjected to compositional analysis of the comprising disaccharide units by capillary electrophoresis. Although there were very low levels of detectable HSGAGs on undifferentiated cells (at day 3), there was a progressive and dramatic increase in the total levels of sulfated HSGAGs with differentiation (FIG. 9).

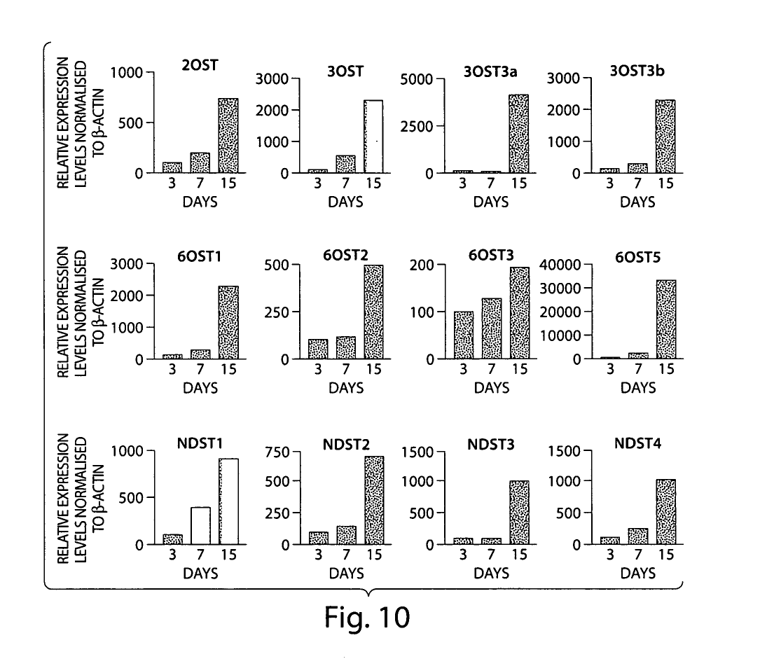

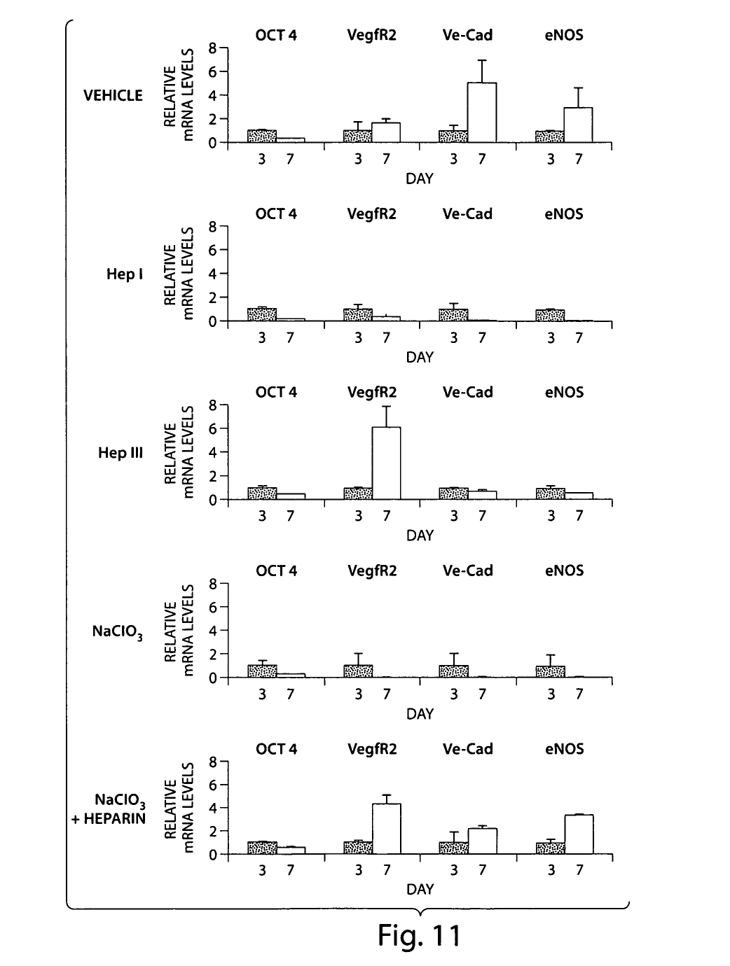

To further confirm these results, changes in the genetic expression of some critical HSGAG biosynthetic enzymes during differentiation were investigated. Biosynthesis of HSGAGs in mammals is initiated by the formation of a tetrasaccharide linkage (Glucuronic Acid-Galactose-Galactose-Xylose) to a proteoglycan core (Varki, et al. (1999) Essentials of Glycobiology, Cold Spring Harbor, N.Y.). After the initial formation of this linkage tetrasaccharide, the alternating addition of glucuronic acid and N-acetyl-glucosamine from their UDP-sugar nucleotide precursors forms a repeating 1,4-linked disaccharide chain. The disaccharide chain is further modified by a series of sulfotransferases, of which N-deacetylase-N-sulfotransferase and the 2-O, 3-O, and 6-O heparan sulfate sulfotransferases play a role. Tissue and substrate specific isoforms of many of these sulfotransferases have been discovered, indicating a further level of complexity in the biosynthesis of HSGAGs (Lindahl, U., et al. (1998) J Biol Chem 273, 24979-82; Habuchi, O. (2000) Biochim Biophys Acta 1474, 115-27). It is the sulfotransferases that give HSGAGs their “signature” structure, and thus these enzymes are critical in modulating specific structure-function relationships of HSGAGs (Sasisekharan, R., and Venkataraman, G. (2000) Curr Opin Chem Biol 4, 626-31). Real-time PCR analysis was performed for 2-O, 3-O and 6-O sulfotransferases, N-deacetylase-N-sulfotransferase, and their isoforms. This analysis demonstrated that there were significantly higher levels of all sulfotransferase transcripts as differentiation progressed with time, supporting the results from the compositional analysis of HSGAGs (FIG. 10). These results also suggest a role for HSGAGs in differentiation of ES cells.

HSGAGs Modulate Differentiation of ES cells into Endothelial Cells

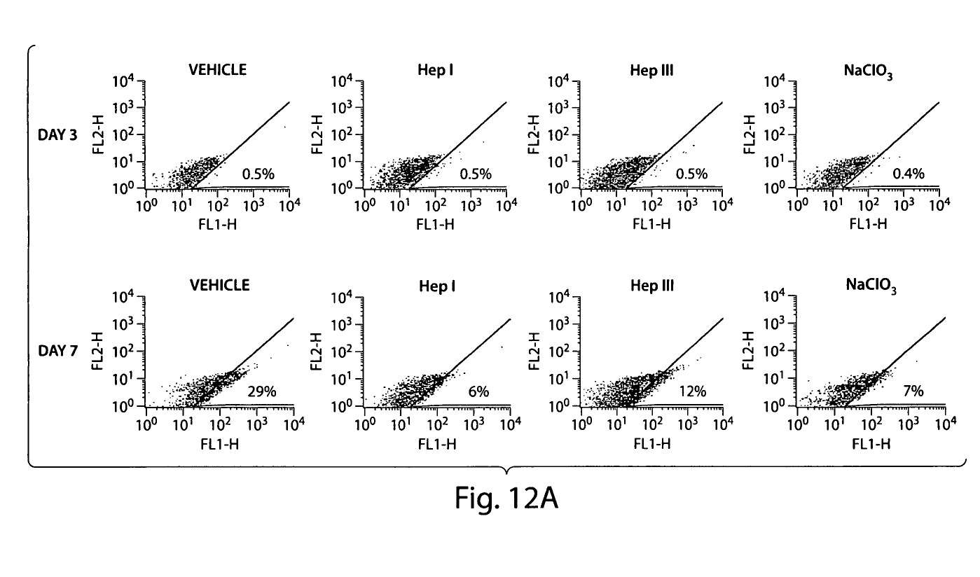

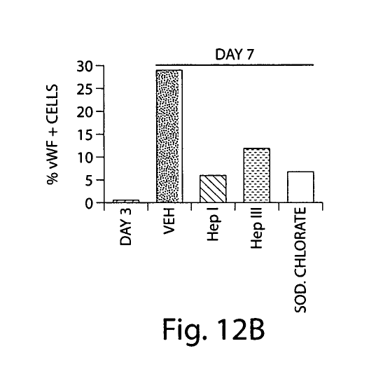

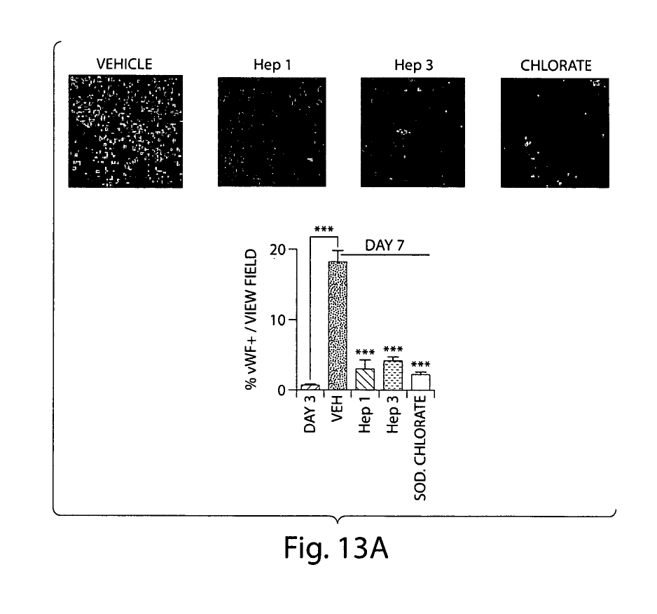

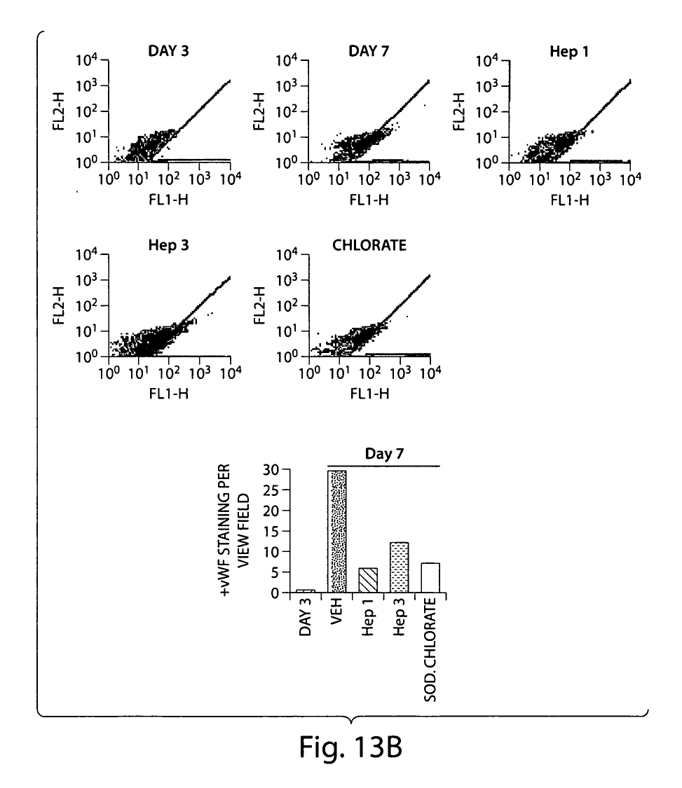

To dissect the role of HSGAG structure in ES cell differentiation, the cell surface and extracellular HSGAG moieties of differentiating ES cells were modified using an enzymatic and pharmacological approach. Specifically, differentiating ES cells were incubated with Hep-I or Hep-III for 30 minutes every day to cleave the cell surface and extracellular matrix (ECM) HSGAGs at structurally distinct sites, or cultured in media supplemented with sodium chlorate, which is a pharmacological inhibitor of HSGAG biosynthesis (Safaiyan, F., et al. (1999) J Biol Chem 274, 36267-73). Hep-I cleaves HSGAGs at highly sulfated regions, and Hep-III cleaves HSGAGs at undersulfated regions (Linhardt, R. J., et al. (1990) Biochemistry 29, 2611-7; Godavarti, R., and Sasisekharan, R. (1996) Biochem Biophys Res Commun 229, 770-7). At the end of enzymatic degradation, the medium containing the enzymes and HSGAG fragments was replaced with fresh medium. At different time points, the differentiation of ES cells into endothelial cells was monitored using flow cytometry, confocal microscopy and real-time PCR. Flow cytometry analysis revealed that all the treatments inhibited the expression of vWF factor, although to different extents (FIG. 12A). Hep-I, which cleaves the HSGAGs at highly sulfated regions, and sodium chlorate, which inhibits HSGAG biosynthesis, both decreased the proportion of cells expressing detectable levels of vWF by approximately 5 fold. Hep-III, which cleaves the HSGAGs at undersulfated regions, although milder, also showed an inhibitory effect by ˜3 fold (FIG. 12B).

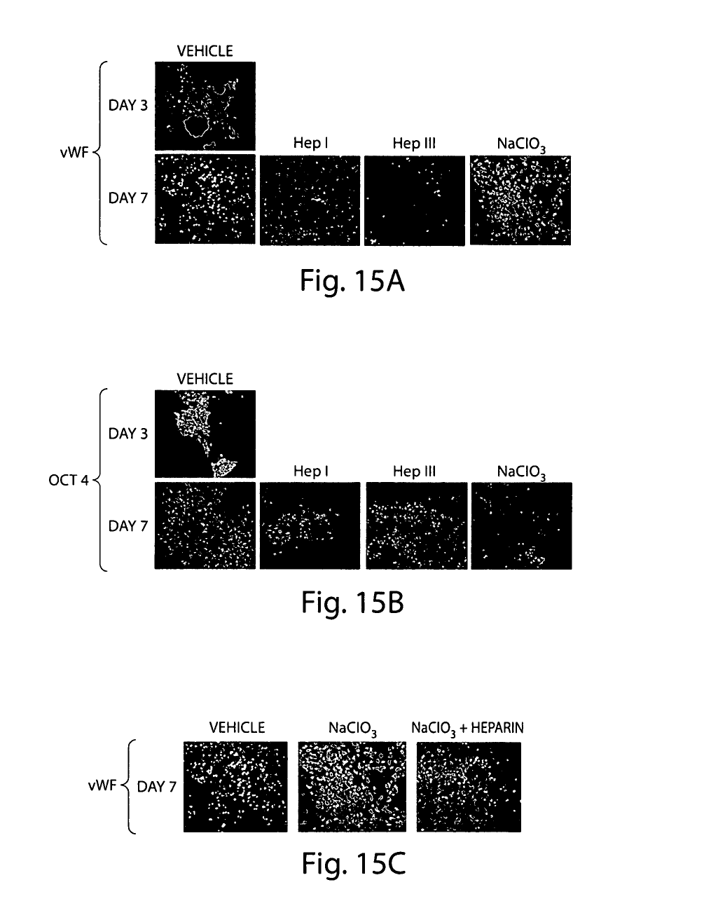

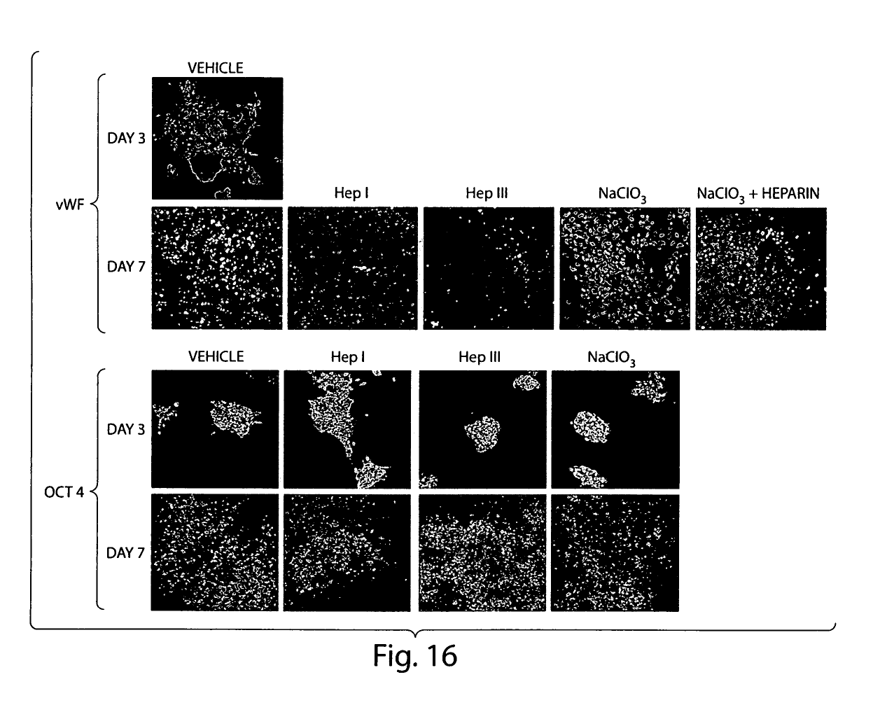

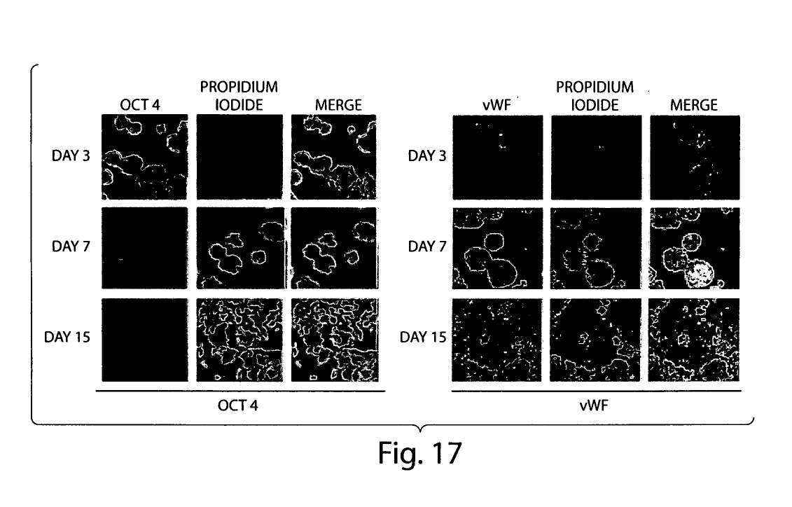

The inhibitory effects of Hep-I, Hep-III and sodium chlorate treatments on vWF expression were also confirmed by confocal microscopy studies (FIG. 15A). Interestingly, none of the treatments had significant effects on Oct-4 levels of differentiating cells (FIG. 15B). These findings suggest that treatment with Hep-I, Hep-III or sodium chlorate does not inhibit differentiation of ES cells, but specifically inhibits their differentiation into endothelial cells. Most importantly, addition of heparin to sodium chlorate treated ES cells during differentiation reconstituted conditions that favor differentiation towards endothelial cells as detected by increased vWF staining (FIG. 15C).

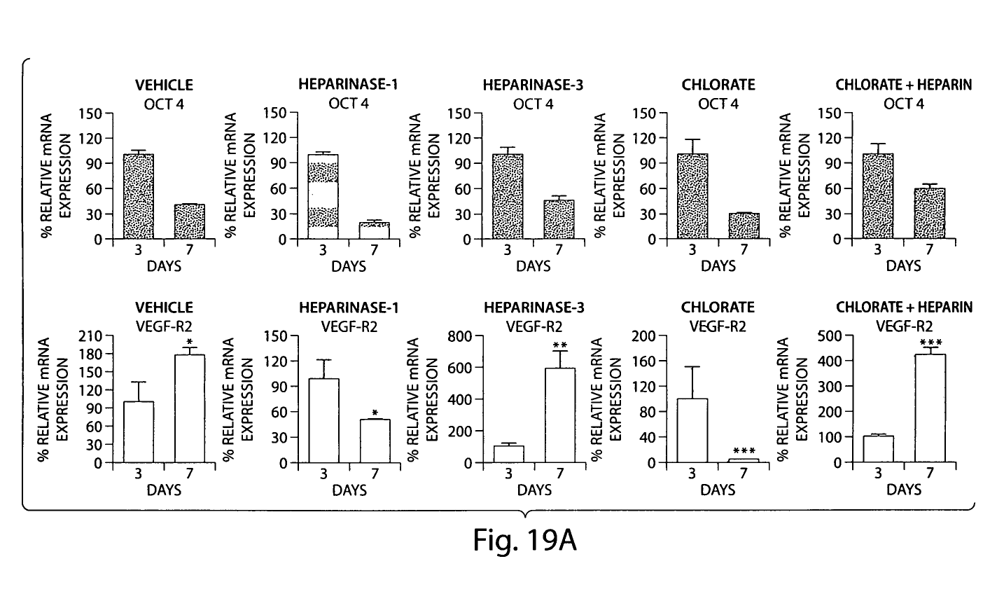

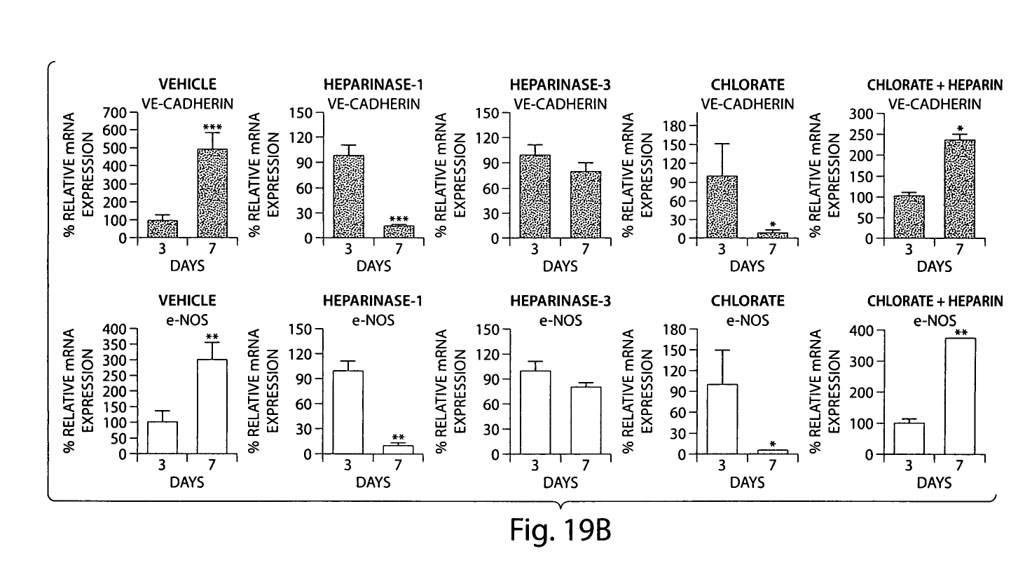

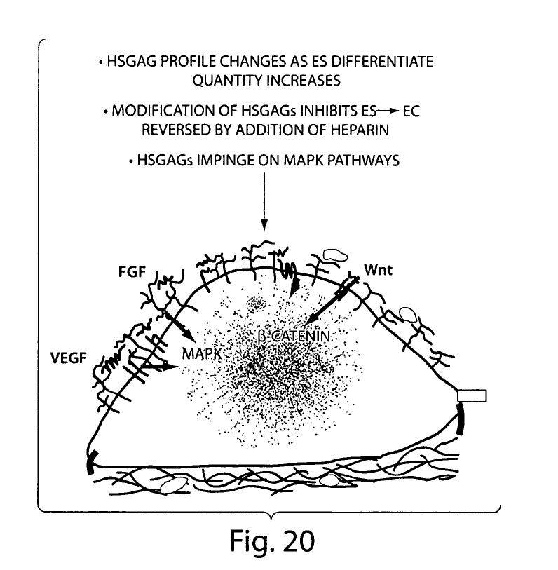

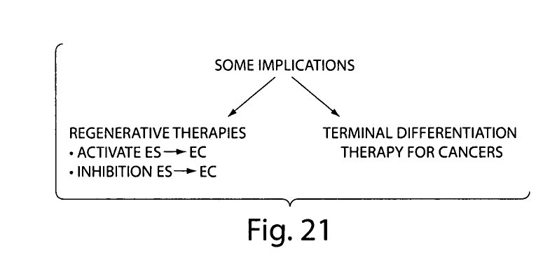

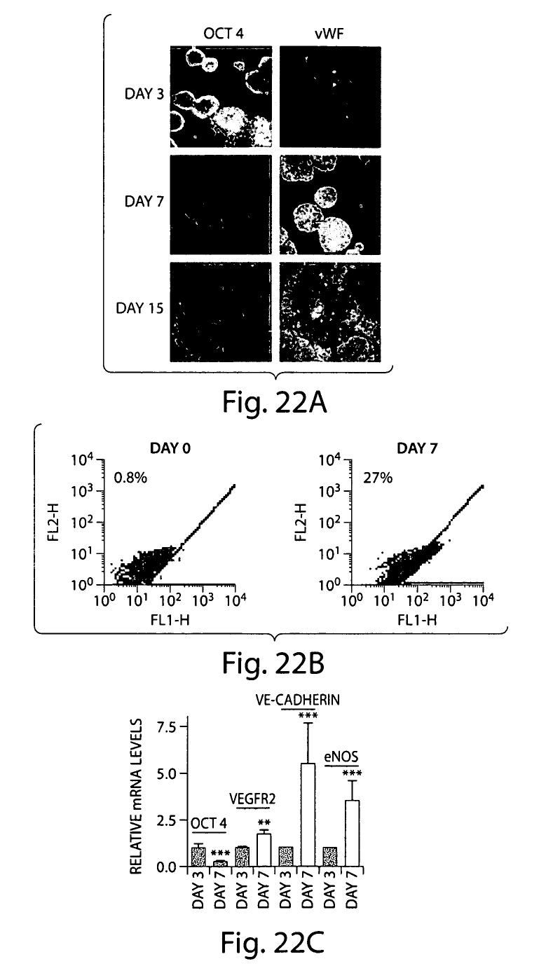

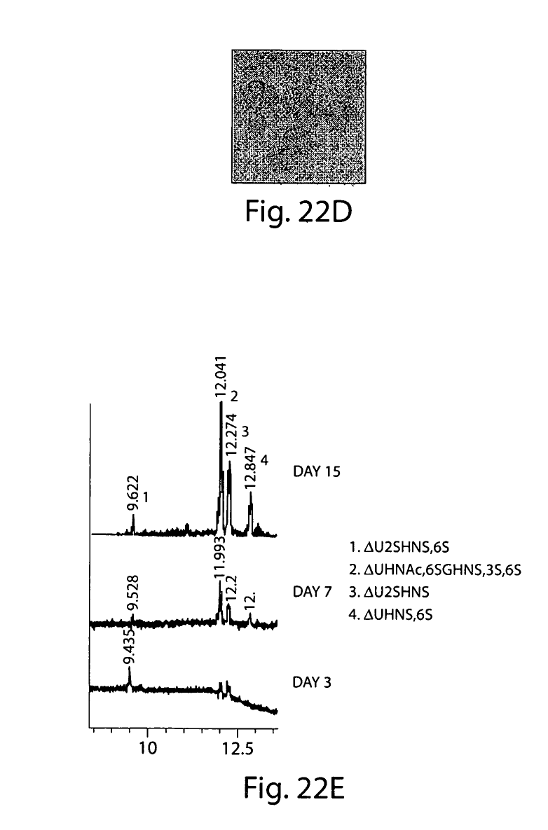

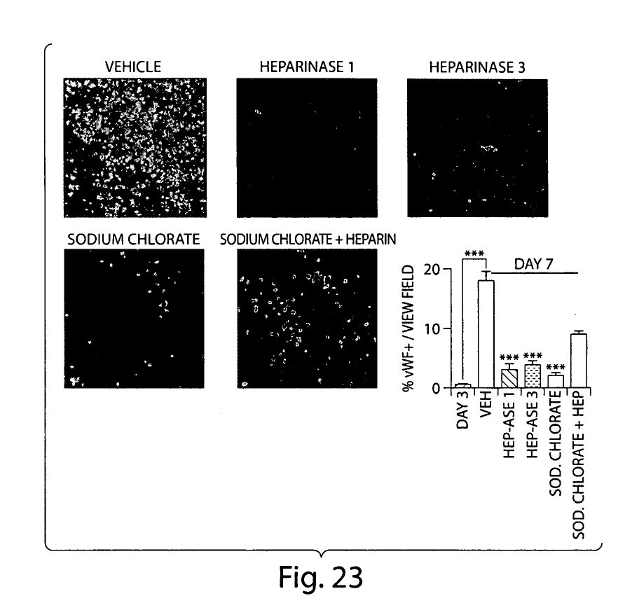

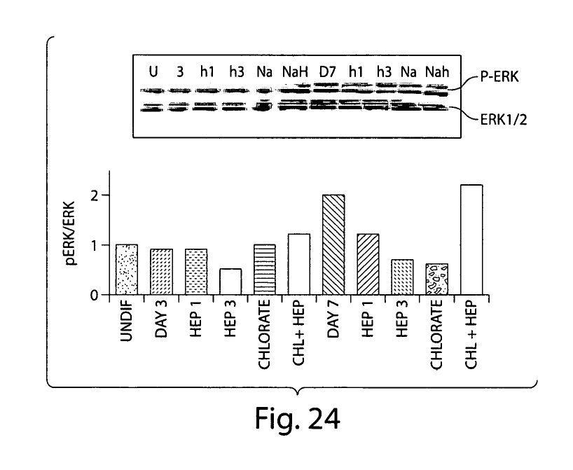

Real-time PCR experiments further confirmed these findings. While there was no effect on Oct-4 transcript levels with any of the treatments, Hep-I and sodium chlorate treatments significantly decreased the transcript levels of VEGF-R2, VE-cadherin and eNOS (FIG. 19). Hep-III treatment also decreased the transcript levels of VE-cadherin and eNOS, however, surprisingly increased the transcript levels of VEGF-R2 (FIG. 19). Orthogonal effects of Hep-I and Hep-Ill in cell phenotype have been reported elsewhere (Liu, D., et al. (2002) Proc Natl Acad Sci USA 99, 568-73), these results suggest that HSGAGs modulate differentiation of ES cells into endothelial cells in a structurally specific manner. Consistent with the confocal microscopy results, real-time PCR results also show that addition of heparin to sodium chlorate treated ES cells reconstituted conditions that favor differentiation towards endothelial cells as detected by increased VEGF-R2, VE-cadherin and eNOS transcript levels.

HSGA Gs Impinge on ES cell Differentiation via MAPK Pathway

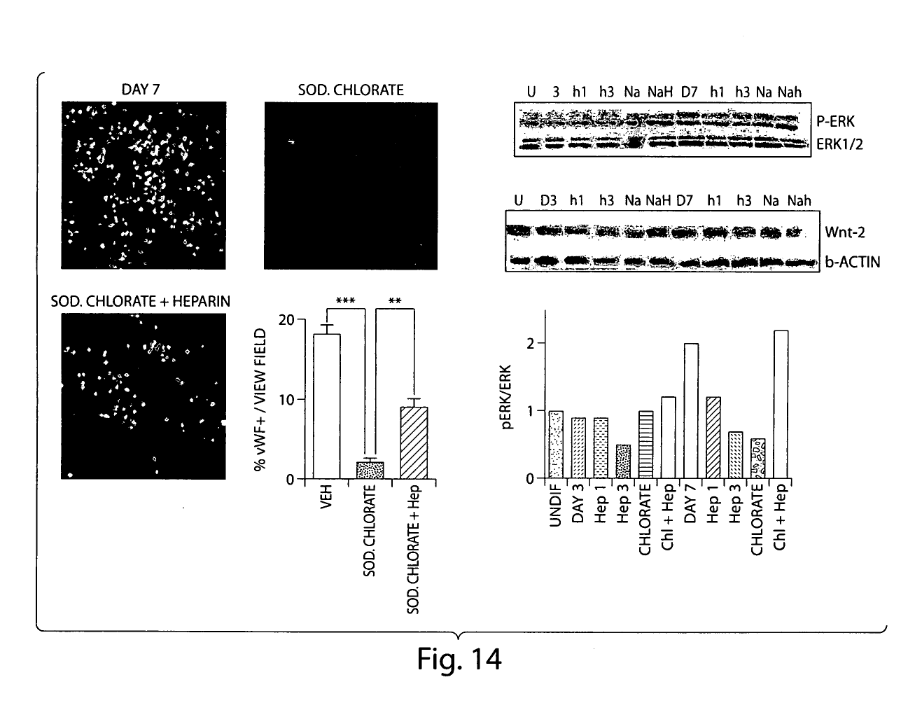

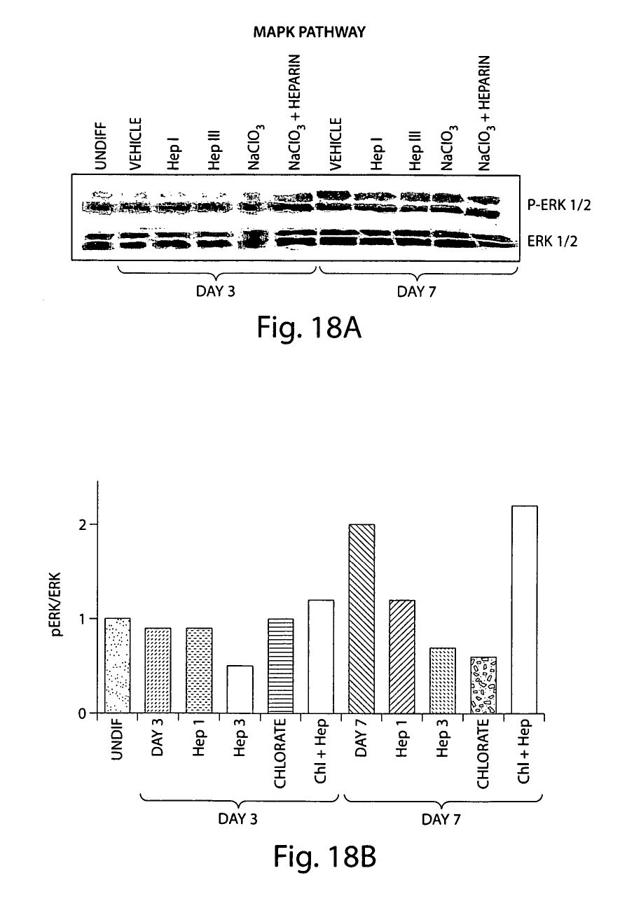

The MAPK pathway is the downstream convergence point of the signaling of several angiogenic factors such as VEGF, FGF, HGF, EGF, PDGF and angiopoietins (Sengupta, S., et al. (2003) Arterioscler Thromb Vasc Biol 23, 69-75). Given the role of these factors in endothelial cell proliferation (Le Querrec, A., et al. (1993) Baillieres Clin Haematol 6, 711-30), it was investigated whether HSGAG modulation was impinging on the MAPK pathway of differentiating ES cells. As shown in FIGS. 14 and 18, treatment of differentiating ES cells with HSGAG-modifying enzymes, Hep-I or Hep-III, or the pharmacological inhibitor, sodium chlorate, inhibits the phosphorylation of ERK1/2. Interestingly this inhibition is reversed by the addition of exogenous heparin. These results suggest that MAPK pathway is involved in differentiation of ES cells, and HSGAGs are modulators of this pathway.

Discussion

Neovascularization is involved in many physiological processes such as development, reproduction, tissue regeneration and wound healing, and is highly regulated through an ‘angiogenic switch’ (Folkman, J. (1997) Exs 79, 1-8; Zetter, B. R. (1988) Chest 93, 159S-166S). However, aberrant neovascularization underlies many pathophysiological conditions. For example, in diabetes, hypercholesterolemia and advanced age, dysfunctional endothelial cells and impaired neovascularization can result in ischemic tissue (Rivard, A., et al. (1999) Circulation 99, 111-20; Rivard, A., et al. (1999) Am J Pathol 154, 355-63; Van Belle, E., et al. (1997) Circulation 96, 2667-74). This can lead to chronic wounds and cause the loss of extremities in over 3% of all diabetics, or result in impaired cardiovascular function in coronary artery disease. For such cases, endothelial cell regeneration and transplantation has potential therapeutic implications in treating patients.

It has been demonstrated that embryonic stem cells exhibited the potential to differentiate into endothelial cells and form vessel-like structures in vitro and in vivo (Levenberg, S., et al. (2002) Proc Natl Acad Sci USA 99, 4391-6). Herein, endothelial specific markers, such as vWF, VE-cadherin, eNOS and VEGF-R2 were used to follow the differentiation of ES cells into endothelial cells over a period of 7 to 15 days. Both confocal microscopy and flow cytometry analysis suggested that the stems cells progressively differentiate into endothelial cells, which was supported by real-time PCR data. In addition, the formation of cord-like primordial vascular structures by confocal microscopy by day 10 was observed, indicating that the embryoid bodies serve as an interesting model to study the molecular mechanisms of vasculogenesis and early angiogenesis.

Although differentiation of embryonic stem cells into endothelial cells has been suggested previously, limited knowledge existed about the possible factors that impinge on this differentiation process. Other studies that attempted to elucidate these factors and the underlying mechanisms have primarily been based on transcriptomic and proteomic approaches. Provided herein, the role of HSGAGs in ES cell differentiation into endothelial cells was dissected. The experiments provide that the modulation of the HSGAG moiety of differentiating ES cells, by either enzymatic degradation or by inhibition of their biosynthesis, inhibits their differentiation into endothelial cells. Interestingly, low levels of HSGAGs on the surface of undifferentiated ES cell were detected, and there was a progressive increase in the highly sulfated HSGAG signal with differentiation. Altogether, these findings suggest that the HSGAG components play a role in determining which cell lineage the differentiation process will yield.

To dissect the possible signaling pathways impinged by HSGAGs of differentiating ES cells, the effect of HSGAG modulation on MAPK pathway was also studied. MAPK is a key convergence point in the signal transduction pathways of multiple angiogenic factors, including tyrosine kinase receptor ligands, such as FGF, VEGF, HGF, EGF, PDGF and angiopoietins (Sengupta, S., et al. (2003) Arterioscler Thromb Vasc Biol 23, 69-75; Griffloen, A. W., and Molema, G. (2000) Pharmacol Rev 52, 237-68). The potential for some of these factors to promote the differentiation of stem cells into endothelial cells has been described (Keller, G. M. (1995) Curr Opin Cell Biol 7, 862-9; Darland, D. C., and D'Amore, P. A. (2001) Curr Top Dev Biol 52, 107-49; Hirashima, M., et al. (1999) Blood 93, 1253-63), and all these factors can use HSGAGs as secondary ligand binding sites (Keiser, N., et al. (2001) Nat Med 7, 123-8). The studies described herein show the inhibition of ERK phosphorylation following treatment with HSGAG-degrading enzymes or sodium chlorate. This indicates that HSGAGs are modulators of the upstream MAPK pathway in differentiation of ES cells into endothelial cells. Without being bound by any particular theory, it is possible that paracrine or autocrine signaling through the growth factors, which act through the MAPK pathway, could lead to endothelial cell enrichment as a result of differentiation. Intriguingly, the inhibition of the MAPK pathway has been described as promoting the self renewal of embryonic stem cells (Qi, X., et al. (2004) Proc Natl Acad Sci USA 101, 6027-32). The MAPK pathway plays a role in stem cell differentiation, and the use of pharmacological inhibitors, such as PTK787 against VEGFR or antibodies against FGFR, to selectively knock out signaling pathways could be used for further study.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

I/We claim:

1. A method of modulating stem cell differentiation, comprising:
contacting the microenvironment of a stem cell with a glycosaminoglycan (GAG)-modulating agent in an amount effective to modulate stem cell differentiation to endothelial cells.

2. The method of claim 1, wherein the GAG-modulating agent is a GAG-degrading agent.

3.-7. (canceled)

8. The method of claim 1, wherein the GAG-modulating agent is a GAG.

9.-13. (canceled)

14. The method of claim 1, wherein the GAG-modulating agent is expressed by a cell, and the cell is contacted with the stem cell microenvironment.

15.-18. (canceled)

19. The method of claim 1, wherein the GAG-modulating agent is a mammalian biosynthetic or biodegradative enzyme.

20.-24. (canceled)

25. The method of claim 1, wherein the GAG-modulating agent inhibits stem cell differentiation to endothelial cells.

26. The method of claim 1, wherein the GAG-modulating agent promotes stem cell differentiation to endothelial cells.

27.-47. (canceled)

48. A method of modulating stem cell differentiation, comprising:
contacting the microenvironment of a stem cell with an agent that alters the biosynthetic or degradation pathway of GAGs in an amount effective to modulate stem cell differentiation to endothelial cells.

49. The method of claim 48, wherein the agent inhibits or promotes the presence of a GAG.

50.-54. (canceled)

55. A method of producing a population of cells, comprising:
contacting the microenvironment of a stem cell with a GAG-modulating agent to inhibit or promote stem cell differentiation to endothelial cells, and obtaining a population of cells.

56. The method of claim 55, wherein the stem cell is promoted to differentiate to endothelial cells.

57.-58. (canceled)

59. The method of claim 55, wherein the stem cell is inhibited from differentiating to endothelial cells.

60.-62. (canceled)

63. A composition, comprising:
the cell population obtained from claim 55.

64. (canceled)

65. A method of treatment, comprising:
administering a GAG-modulating agent, a cell that expresses the GAG-modulating agent or a composition of claim 63 to a subject in an amount effective to treat the subject.

66.-68. (canceled)

69. The method of claim 65, wherein the subject has cancer, and the GAG-modulating agent, cell that expresses the GAG-modulating agent or composition is in an amount effective to inhibit stem cell differentiation to endothelial cells.

70. The method of claim 65, wherein the subject has a neurodegenerative disorder or nervous system injury, and the GAG-modulating agent, cell that expresses the GAG-modulating agent or composition is in an amount effective to inhibit stem cell differentiation to endothelial cells.

71. The method of claim 65, wherein the subject has a chronic wound, and the GAG-modulating agent or composition is in an amount effective to treat the chronic wound.

72. The method of claim 65, wherein the subject has ischemic tissue.

73. The method of claim 65, wherein the subject has diabetes, hypercholesterolemia, coronary artery disease or arthritis.

74. The method of claim 65, wherein the subject is of advanced age.

75.-80. (canceled)


SEQUENCE LISTING (12 sequences)

SEQ ID NO: 1 - DNA, 20 bp, Artificial sequence
  Synthetic oligonucleotide
ccaatcagcttgggctagag

SEQ ID NO: 2 - DNA, 20 bp, Artificial sequence
  Synthetic oligonucleotide
ctgggaaaggtgtccctgta

SEQ ID NO: 3 - DNA, 20 bp, Artificial sequence
  Synthetic oligonucleotide
accgagagaaacaggctgaa

SEQ ID NO: 4 - DNA, 20 bp, Artificial sequence
  Synthetic oligonucleotide
agacggggaagttgtcattg

SEQ ID NO: 5 - DNA, 20 bp, Artificial sequence
  Synthetic oligonucleotide
ggacagtgctccaaccaaat

SEQ ID NO: 6 - DNA, 20 bp, Artificial sequence
  Synthetic oligonucleotide
gttcacactgcagacccaga

SEQ ID NO: 7 - DNA, 20 bp, Artificial sequence
  Synthetic oligonucleotide
gctttcggtagtgggatgaa

SEQ ID NO: 8 - DNA, 20 bp, Artificial sequence
  Synthetic oligonucleotide
ggccttccatttctgtacca

SEQ ID NO: 9 - DNA, 20 bp, Artificial sequence
  Synthetic oligonucleotide
tcttcgttcagccatcacag

SEQ ID NO: 10 - DNA, 20 bp, Artificial sequence
  Synthetic oligonucleotide
cctatagcccgcatagcgta

SEQ ID NO: 11 - DNA, 20 bp, Artificial sequence
  Synthetic oligonucleotide
agccatgtacgtagccatcc

SEQ ID NO: 12 - DNA, 20 bp, Artificial sequence
  Synthetic oligonucleotide
ctctcagctgtggtggtgaa